(19) United States
(12) Patent Application Publication
(10) Pub. No.: US 2020/0352905 A1
(43) Pub. Date: Nov. 12, 2020

(54) 6-(2,4-DICHLOROPHENYL)-5-[4-[(3S)-1-(3-FLUOROPROPYL)PYRROLIDIN-3-YL]OXYPHENYL]-8,9-DIHYDRO-7H-BENZO[7]ANNULENE-2-CARBOXYLIC ACID FOR USE IN METASTATIC OR ADVANCED BREAST CANCER PATIENTS

(71) Applicant: SANOFI, Paris (FR)

(72) Inventors: Sylvaine CARTOT-COTTON, Paris (FR); Marina CELANOVIC, Bridgewater, NJ (US); Patrick COHEN, Paris (FR); Gautier PAUX, Bridgewater, NJ (US)

(73) Assignee: SANOFI, Paris (FR)

(21) Appl. No.: 16/870,031

(22) Filed: May 8, 2020

Related U.S. Application Data
(60) Provisional application No. 62/945,311, filed on Dec. 9, 2019.

(30) Foreign Application Priority Data
May 9, 2019 (EP) 19305593.6
May 28, 2019 (EP) 19305679.3

(51) Int. Cl. A61K 31/40; A61J 1/00

(52) U.S. Cl. CPC A61K 31/40; A61K 45/06; A61J 1/00

(57) ABSTRACT

The compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, for use in the treatment of metastatic or advanced breast cancer at a dose of 150 to 600 mg per day.

Herein is provided the compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, for use in the treatment of metastatic or advanced breast cancer at a dose of 150 to 600 mg per day.

The estrogen receptor α (ESR1) is expressed in the majority of breast tumors, enabling them to respond to the mitogenic actions of estrogens.

6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, hereafter designated as “compound (1)”, is represented below:

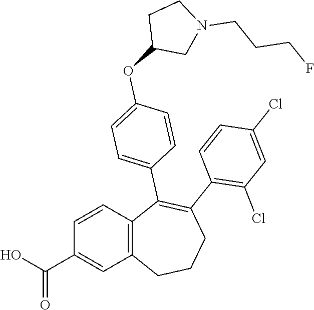

This compound is a selective estrogen receptor degrader (SERD) which has complete estrogen receptor antagonist properties and accelerates the proteasomal degradation of the estrogen receptor. It is disclosed in the international patent application WO 2017/140669, along with its therapeutic properties, including breast cancer.

It has now been found that compound (1) may be used in a human patient at a dose of 150 to 600 mg per day for the treatment of metastatic or advanced breast cancer, which are the most serious stages of cancer (late stage). Advanced breast cancer is herein defined as a cancer wherein the tumor is not in a local regional area (i.e., it is outside of the primary tumor location), or if it is it cannot be removed by surgery. Metastatic breast cancer is defined herein as a cancer which has spread to other sites of the body, such as the liver, lungs, bones, brain, and/or others.

In an embodiment, compound (1) or a pharmaceutically acceptable salt thereof is used in the treatment of metastatic or advanced breast cancer at a dose of 400 to 600 mg per day, more particularly at a dose of 400 mg per day.

Provided herein is also a pharmaceutical composition comprising 150 to 600 mg of compound (1), or a pharmaceutically acceptable salt thereof, as well as at least one pharmaceutically acceptable excipient. In an embodiment, the pharmaceutical composition comprises 400 to 600 mg of compound (1), or a pharmaceutically acceptable salt thereof, as well as at least one pharmaceutically acceptable excipient. In another embodiment, the pharmaceutical composition comprises 400 mg of compound (1), or a pharmaceutically acceptable salt thereof, as well as at least one pharmaceutically acceptable excipient. The pharmaceutically acceptable excipients are selected from the customary excipients which are known to a person skilled in the art. More particularly, the excipients are selected from those useful for oral administration in whatever form (liquid solution, dispersion or suspension, tablets, capsules or the like).

Herein is also provided a method of treating metastatic or advanced breast cancer comprising administering to a patient in need thereof, more particularly a human patient, a pharmaceutical composition as described above.

Herein is also provided a method of treating metastatic or advanced breast cancer, comprising administering to a subject in need thereof a therapeutically effective amount of compound (1), or a pharmaceutically acceptable salt thereof. In another embodiment, the invention relates to a method of treating metastatic or advanced breast cancer, comprising administering to a subject in need thereof compound (1), or a pharmaceutically acceptable salt thereof, at a dose of 150 to 600 mg per day. In another embodiment, the invention relates to a method of treating metastatic or advanced breast cancer, comprising administering to a subject in need thereof compound (1), or a pharmaceutically acceptable salt thereof, at a dose of 400 to 600 mg per day, more particularly at a dose of 400 mg per day.

Herein is also provided an article of manufacture, a packaging, or an administration unit, comprising:
a packaging material;
the above defined pharmaceutical composition; and
a label or package insert contained within said packaging material, indicating that said pharmaceutical composition is administered to a patient for the treatment of metastatic or advanced breast cancer, at a dose of 150 to 600 mg per day of compound (1) or a pharmaceutically acceptable salt thereof, more particularly at a dose of 400 to 600 mg per day, even more particularly at a dose of 400 mg per day.

Herein is also provided a dose of 150 to 600 mg of compound (1), or a pharmaceutically acceptable salt thereof, for the manufacture of a medicament useful in treating metastatic or advanced breast cancer. In another embodiment, herein is provided a dose of 400 to 600 mg of compound (1), or a pharmaceutically acceptable salt thereof, for the manufacture of a medicament useful in treating metastatic or advanced breast cancer. In another embodiment, herein is provided a dose of 400 mg of compound (1), or a pharmaceutically acceptable salt thereof, for the manufacture of a medicament useful in treating metastatic or advanced breast cancer.

In another embodiment, compound (1), or a pharmaceutically acceptable salt thereof, is administered by the oral route.

The doses described herein are expressed as net doses, namely total doses of the active ingredient per day and per patient. Such net doses may be administered in one or more times during a 24-hour period, more particularly in one time or in two times spread out over time.

In another embodiment, the breast cancer is an estrogen receptor positive cancer (ER positive breast cancer).

In another embodiment, the breast cancer is a human epidermal growth factor receptor 2 (HER2) negative cancer.

In another embodiment, the breast cancer is an estrogen receptor positive, human epidermal growth factor receptor 2 negative cancer.

In another embodiment, the breast cancer is an advanced one.

In another embodiment, the breast cancer is a metastatic one.

In another embodiment, the cancer is a cancer with wild type estrogen receptors.

In another embodiment, the cancer is a cancer with deregulated function of estrogen receptors related to, but not limited to, at least one epigenetic and genetic alteration of estrogen receptors such us mutation, amplification, splice variant.

In another embodiment, the cancer is a cancer with mutated estrogen receptors.

In another embodiment, the patient is a woman.

In another embodiment, the patient is a postmenopausal woman.

In another embodiment, the patient is a premenopausal woman on menopause-inducing medication; said medication is typically a gonadotropin-releasing hormone, more particularly a luteinizing releasing hormone agonist, such as buserelin or goserelin.

In another embodiment, the patient is treated with compound (1) as a second, third or subsequent line treatment.

In another embodiment, the patient has been pretreated with 1 to 8 anti-cancer treatments for metastatic or advanced breast cancer. In another embodiment, the patient has been pretreated with 1 to 6 anti-cancer treatments for metastatic or advanced breast cancer. In another embodiment, the patient has been pretreated with 2 anti-cancer treatments for metastatic or advanced breast cancer.

In another embodiment, the patient has been pretreated with at least one endocrine therapy treatment for metastatic or advanced breast cancer. Common classes of drugs used as endocrine therapy include selective estrogen receptor modulator (SERM) such as tamoxifen; aromatase inhibitors (AI) such as letrozole, anastrozole or exemestane; and selective estrogen receptor down-regulators (SERD) such as fulvestrant.

More particularly, the patient demonstrates disease progression or recurrence after said endocrine therapy.

In another embodiment, the patient has been pretreated for at least 6 months with said endocrine therapy.

In another embodiment, the patient has been pretreated with at least one endocrine therapy treatment for metastatic or advanced breast cancer, as well as with at least one chemotherapy and/or targeted therapy, such as a tyrosine kinase inhibitor, including mTOR and CDK4/6 inhibitors.

In yet another embodiment, the patient has been pretreated with at least one endocrine therapy treatment for metastatic or advanced breast cancer, as well as with at least one but no more than three (i.e. one to three) chemotherapies and/or one or more targeted therapies.

In yet another embodiment, the patient has been pretreated with at least one endocrine therapy treatment for metastatic or advanced breast cancer, as well as with at least one but no more than three (i.e. one to three) chemotherapies and/or one or more targeted therapies, with a range of 1 to 8 prior anti-cancer treatments.

In yet another embodiment, combined with any of the embodiments described herein, the patient has been pretreated with no more than one treatment with a CDK4/6 inhibitor.

Chemotherapy is herein defined as an anti-cancer drug acting in a non-specific way by inhibiting mitosis (cell division), hence acting as a cytotoxic treatment.

Endocrine therapy, or hormonal therapy, is defined herein as a treatment with specific genetic targets, which inhibit growth-promoting signals from hormones, especially endocrine hormones (estrogens for breast cancer). By contrast, inhibition of growth-signals like those associated with receptor tyrosine kinases are referred to as targeted therapy, which blocks the growth of cancer cells by interfering with specific targeted molecules needed for carcinogenesis and tumor growth.

In another embodiment, the use of compound (1) in the treatment of metastatic or advanced breast cancer at a dose of 150 to 600 mg per day, in heavily pretreated patients (with targeted therapy and prior fulvestrant, more specifically patients with a median of 2 prior anti-cancer therapies (range 1-8) in the advanced setting), provides a Clinical Benefit Rate which is similar, based on an indirect literature comparison, to the one observed with fulvestrant when administered to patients with no prior targeted therapy neither prior fulvestrant treatment. In another embodiment, said Clinical Benefit Rate for compound (1) is about 36%, more specifically 35.6%. Herein, the Clinical Benefit Rate (CBR) is defined as the percentage of patients who have achieved CR (Complete Response), PR (Partial Response) and prolonged SD (Stable Disease for 24 weeks or more) to the treatment with compound (1). It represents patients whose tumors either shrink or remain stable under said treatment.

In another embodiment, the use of compound (1) in the treatment of metastatic or advanced breast cancer at a dose of 150 to 600 mg per day provides, in patients with no more than 3 prior lines of anti-cancer therapies, who have received no prior mTOR inhibitor treatment, and have optionally received a prior chemotherapy or a CDK4/6 inhibitor treatment but not both, a Clinical Benefit Rate, defined as above, which is increased, based on an indirect literature comparison, compared to the one observed with fulvestrant when administered to patients with no prior targeted therapies neither prior fulvestrant treatment. In another embodiment, said Clinical Benefit Rate for compound (1) is about 47%, more specifically 46.9%.

In another embodiment, the use of compound (1) in the treatment of metastatic or advanced breast cancer at a dose of 150 to 600 mg per day provides, in patients with neither prior targeted therapies (such as mTOR or CDK4/6 inhibitors) nor prior SERD therapy (such as fulvestrant), a Clinical Benefit Rate, defined as above, which is increased, based on an indirect literature comparison, compared to the one observed with fulvestrant in such patients. In another embodiment, said Clinical Benefit Rate for compound (1) is about 64%, more specifically 64.3%.

All embodiments described above may be combined with any of the features described herein in relation to compound (1) for its therapeutic use in metastatic or advanced breast cancer, to the method of treating metastatic or advanced breast cancer comprising administration of compound (1), to the pharmaceutical composition comprising compound (1), and to the article of manufacture, packaging or administration unit comprising said pharmaceutical composition.

The examples below show the pharmacological and clinical results obtained with compound (1) in metastatic or advanced breast cancer patients.

### DESCRIPTION OF DRAWINGS

FIG. 1: Post-treatment ¹⁸FES-PET scan performed 16-25 hours after the previous treatment administration (10 hours for two patients indicated by *).

### LIST OF ABBREVIATIONS

AEs Adverse events
BID Bis in die (twice daily)
BOR Best Overall Response
CBR Clinical Benefit Rate
CI Confidence interval
COD Cut-off date
CR Complete Response
CT Computerized tomography
CV Coefficient of variation
DL Dose level
DLT Dose-limiting toxicity
eCRF Electronic Case Report Form
ECOG PS Eastern Cooperative Oncology Group (ECOG) Performance Status
ER Estrogen receptor
ESR1 Estrogen receptor 1
FDG Fluorodeoxyglucose
FES Fluoroestradiol
FFPE Formalin-fixed and paraffin-embedded
FSH Follicle stimulating hormone
GnRH Gonadotropin-releasing hormone
HER2 Human epidermal growth factor receptor 2
ICR Independent Central Review
IHC Immunohistochemistry
MAD Maximum administered dose
MedDRA PT Medical Dictionary for Regulatory Activities, Preferred Term
MTD Maximum tolerated dose
NCI-CTCAE National Cancer Institute Common Terminology Criteria for adverse events
ORR Objective Response Rate
PD Pharmacodynamic
PET Positron emission tomography
PK Pharmacokinetics
PR Partial Response
QD Quaque die (once daily)
RD Recommended dose
RECIST Response evaluation criteria in solid tumors
SAEs Serious adverse events
SD Stable disease
SUV Standardized uptake value
TEAEs Treatment-emergent adverse events

1: Protocol

A Phase 1/2 Study for the Safety, Efficacy, Pharmacokinetic and Pharmacodynamics Evaluation of Compound (1), Administered Orally as Monotherapy in Postmenopausal Women with Estrogen Receptor-Positive Advanced Breast Cancer (TED14856).

The goals of this study are to evaluate the safety profile, the efficacy, the pharmacokinetics (PK) and the pharmacodynamics (PD) of escalating doses of compound (1). This is an open-label, non-comparative phase 1/2 study, composed of several parts; herein we focus on the first two parts of the study (dose escalation and dose expansion parts) directed to compound (1) administered as monotherapy.

1-1: Study Objectives

Primary Objectives:

Dose escalation (part A): To assess the incidence rate of dose-limiting toxicity (DLT) and to determine the maximum tolerated dose (MTD) as well as the recommended dose (RD) of compound (1) administered as monotherapy to postmenopausal women with estrogen receptor (ER) positive and human epidermal growth factor receptor 2 (HER2) negative advanced breast cancer.

Dose expansion (part B): To assess antitumor activity using objective response rate (ORR) according to response evaluation criteria in solid tumors (RECIST) v1.1 at the compound (1) RD administered as monotherapy, determined by ICR, in postmenopausal women with ER-positive and HER2-negative advanced breast cancer. RECIST (Response Evaluation Criteria In Solid Tumors) is a set of published rules that define when tumors in cancer patients improve (“respond”), stay the same (“stabilize”), or worsen (“progress”) during treatment.

Secondary Objectives:
To characterize the overall safety profile of compound (1).
To characterize the pharmacokinetic (PK) profile of compound (1).
To evaluate the antitumor activity using ORR according to RECIST v1.1 of compound (1) administered as monotherapy in Part A and the clinical benefit rate (CBR, complete response [CR], partial response [PR] and stable disease [SD]≥24 weeks) in Parts A and B.
To evaluate the ORR and the CBR (CR, PR and SD ≥24 weeks) in Part B according to the estrogen receptor 1 (ESR1) gene mutational status (mutant and wild type) at baseline and during treatment.
To evaluate the time to first tumor response (CR or PR) in Part B.
To evaluate residual ER availability with Positron Emission Tomography (PET) scan [(18)F]fluoroestradiol (¹⁸FES) uptake with increasing doses of compound (1) (hereafter “¹⁸FES-PET”) in Part A.
To assess the food effect on PK of compound (1) in Part A.
To assess potential induction/inhibition effect of compound (1) on CYP3A (cytochrome P450 3A) using 4β-OH cholesterol (Parts A and B).

Exploratory Objectives:
To evaluate PK/pharmacodynamics (PD) relationships.
To evaluate target engagement: confirm the ER degradation with re-biopsy of the tumor at recommended dose in at least 10 patients (Part B).
To determine gene mutational profiles of tumor over time (baseline and end of treatment [EOT]) by circulating free DNA (cfDNA) analysis.
To evaluate other breast cancer biomarkers in tumor over time such as Ki67, Bcl-2, PgR, ET and tumor gene expression profiles (Parts A and B).
To assess the extent of metastases with FDG (fluorodeoxyglucose) PET/CT (Computerized Tomography) during dose escalation (Part A).

1-2: Study Design

This is an open-label, non-comparative, dose escalation and dose expansion, safety, efficacy, PK and PD evaluation study of compound (1) administered orally as monotherapy. Compound (1) is given daily to postmenopausal women with ER-positive and HER2-negative advanced breast cancer.

Part A: Dose escalation study to evaluate the safety, PK and PD of compound (1) administered as monotherapy, including pilot food effect sub-study.

Part B: Dose expansion study to evaluate the efficacy, safety, PK and PD of compound (1) administered at the RD (from Part A).

A study committee is set up, including the main Investigators, Sponsor clinical team and ad-hoc experts when appropriate. The study committee decides on whether to escalate (or not) to the next dose level during study committee meetings on the basis of their knowledge of the whole safety profile, ¹⁸FES-PET results (Part A only), and PK profile.

A) Dose Escalation (Part A)

Dose escalation is initiated with a once daily (QD) schedule with a starting dose of 20 mg/day. Dose escalation is expected to proceed according to the Table A below. Intra-patient dose escalation is not permitted.

TABLE A
compound (1) dose levels in Part A

| | Compound (1) |
|---|---|
| Dose level (DL)^{a} | (mg) |
| DL(−1) QD | 10 once daily |
| DL1 QD | 20 once daily |
| DL1bis QD | 50 once daily |
| DL2 QD | 100 once daily |
| DL2bis QD | 150 once daily |
| DL3 QD | 200 once daily |
| DL4 QD | 400 once daily |
| DL4bis BID | 200 twice daily |
| DL5 QD | 600 once daily |
| DL5bis BID | 300 twice daily |

^{a}Additional intermediate or higher dose levels can be tested after agreement between Sponsor and Investigators (study committee). A BID schedule of administration may be added during the study, the starting dose will be a DL of the same dose intensity as the highest cleared DL with QD Schedule. Other schedules of administration may be added during the study.

Part A of this study is designed using the 3+3 concept: 3 to 6 patients are treated at each dose level, depending on DLTs observed in the first 3 patients. If one of the first 3 evaluable patients experiences DLT during Cycle 1, this cohort is expanded with a total of up to 6 patients. If less than 1 out of 3 patients or less than 2 out of 6 patients experience DLTs at a given dose level, the dose escalation is proceeded to the next dose level.

In addition, ¹⁸FES-PET scan should be available for all DLT evaluable patients and depending on results at DL1 and DL2, the intermediate dose levels (DL1bis and DL2bis) could be explored. From these two dose levels DL1bis and DL2bis, the next dose levels (DL2 and DL3 respectively) shall not be skipped.

At subsequent dose levels (≥DL3), other intermediate or higher dose levels may be tested based on safety, ¹⁸FES-PET scan results (if all patients have >90% of inhibition of the target) and PK parameters upon recommendation from the study committee.

The second and third patients of a given cohort are only enrolled when the first patient has received at least 1 week of compound (1) without DLT. The enrolment at the next dose level cannot proceed before at least 3 patients treated at the current dose level have been followed for at least 1 cycle duration (i.e., 28 days) and are evaluable for DLT assessment.

Patients who discontinue study treatment prematurely before the end of DLT observation period for any reason other than DLT are replaced.

As a rule, the dose escalation stops when the maximum administered dose (MAD) is reached, MAD being defined as the dose at which 33% (2 patients out of up to 6) of evaluable patients have experienced a DLT at Cycle 1.

The MTD is defined as the highest dose level at which no more than 1 patient of a maximum of 6 patients experienced a DLT. Usually, the MTD is one dose level below the MAD or the highest dose tested if the MAD is not reached.

Although the dose escalation process is guided by the safety evaluation during Cycle 1 of treatment, cumulative or irreversible toxicities observed after subsequent administrations are also considered for the dose escalation and the dose selection decisions (i.e., expansion of a given dose level, intermediate dose levels), as well as any other relevant information, upon recommendation from the study committee.

The RD for the expansion cohorts is primarily based on safety data, but also on target saturation, PK and PK/PD data. If the MTD cannot be determined in the absence of DLT at the MAD, PK after repeated administration, level of inhibition of target occupancy measured by ¹⁸FES-PET imaging and PK/PD on ER occupancy as well as any other relevant information, are also taken into account to select the RD and for the decision to expand the study to its further parts. The RD shall be potentially at least 2 dose levels above the dose level showing >90% of inhibition of the target on ¹⁸FES-PET scan at this dose level, unless there are DLTs at this dose, in which case the RD can be any dose where >90% inhibition is reached.

The twice a day (BID) regimen is explored on 6 DLT-evaluable patients at the dose level providing the same dose intensity as the highest cleared QD dose level (600 mg): 300 mg taken two times a day 12 hours apart (i.e. 2×300 mg±1 hour). Other doses such as 200 mg taken two times a day 12 hours apart may be explored. In that case, 6 DLT-evaluable patients are enrolled at this dose level.

Pilot food effect: A pilot food effect is assessed by PK sampling after drug administration with a moderate fat breakfast on Day 3 of Cycle 1 in all patients treated in Part A. All other dosing in Part A is taken in fasted condition. If results from the QD dosing regimen allow conclusions to be drawn, this will not be implemented for other dosing regimens (eg, BID) that are explored.

B) Dose Expansion (Part B)

When the dose escalation phase ends for the QD regimen, the RD is proposed by the study committee for the expansion cohort (Part B) and a total of 78 patients is treated at this RD. An interim analysis based on ORR (by RECIST v1.1) is planned when 45 patients are treated in order to decide, based on preset criteria, if the recruitment of planned additional patients is justified (see statistical considerations). If results in Part A with the BID dosing regimen are of interest in terms of safety, PK, exposure, preliminary efficacy and any other relevant information such as data from patients treated with the QD regimen, and warrants further investigation, a BID regimen can be tested in an additional expansion subpart with a total of 56 patients treated at the recommended BID regimen from Part A. In that case, an interim analysis based on ORR (by RECIST v1.1) is planned when 29 patients are treated in order to decide, based on preset criteria, if the recruitment of planned total patients is justified.

1-3: Study Population

### Inclusion Criteria:

I 01. Patients are postmenopausal women as defined by one of the following:
   a. Women >60 years
   b. Women ≤60 years:
   With spontaneous cessation of menses >12 months prior to registration in the absence of chemotherapy, tamoxifen and toremifene;
   Or with cessation of menses of duration ≤2 months or secondary to hysterectomy, and have follicle stimulating hormone (FSH) level in the postmenopausal range according to institutional standards (or >34.4 IU/L if institutional range is not available) prior to registration;
   Or who have received hormonal replacement therapy but who have discontinued this treatment, and have FSH level in the postmenopausal range according to institutional standards (or >34.4 IU/L if institutional range is not available) prior to registration;
   Or with status post bilateral surgical oophorectomy;
   Or are premenopausal women on a gonadotropin-releasing hormone (GnRH) analog for at least 6 months (to be continued during study treatment) and have a negative pregnancy test prior to initiation of study treatment and at monthly intervals during treatment.
I 02. Patients with histological or cytological proven diagnosis of the breast adenocarcinoma with evidence of either locally advanced not amenable to radiation therapy or surgery in a curative intent, inoperable and/or metastatic disease.
I 03. Either the primary tumor or any metastatic site must be positive for ER (>1% tumor cell staining by immunohistochemistry (IHC).
I 04. Either the primary tumor or any metastatic site must be HER2 non-overexpressing by IHC (0, 1+) or in situ hybridization-negative based on single-probe average HER2 copy number <4.0 signals/cell or dual-probe HER2/centromeric probe for chromosome 17 (CEP17) ratio <2 with an average HER2 copy number <4.0 signals/cell as per the American Society of Clinical Oncology guidelines.
I 05. Prior chemotherapy for advanced/metastatic disease is allowed (Note: Antibody drug conjugates [ADCs] are considered as chemotherapy in this study):
   Patients must have received no more than 3 prior chemotherapeutic regimens in Part A (dose escalation).
   Patients must have received no more than 1 prior chemotherapeutic regimen in Part B (dose expansion).
I 06. Patients with advanced breast cancer must have received at least 6 months of prior endocrine therapy. Patients who relapsed while on adjuvant endocrine therapy that was initiated ≥24 months ago, or relapsed <12 months after completion of adjuvant endocrine therapy, are eligible.
I 07. Age ≥18 years old.
I 08. Measurable lesion by RECIST v1.1.
I 09. The patient is capable of understanding the informed consent and complying with the protocol and has signed the informed consent form (ICF) before any study (specific screening procedures or evaluations).
   Part A only:
I 10. Patient entering the study must agree to undergo:
   a. two ¹⁸FES-PET/computerized tomography (CT) imaging scans, one at baseline and one between Day 11 and Day 15 of study treatment intake, and
   b. two FDG PET/CT, one at baseline and one between Day 11 and Day 15 of study treatment intake before FES-PET, and
   c. Paired biopsies (before treatment and during treatment): for baseline samples, formalin-fixed and paraffin-embedded (FFPE) archived biopsy samples (within past 3 months prior initiation of study treatment) can be used, but preferably fresh biopsies from primary tumor or recurrence or metastasis, are collected. It is recommended that the end of cycle 2 biopsy be collected at the same location as the baseline biopsy, whenever possible the tumor is accessible for a biopsy during treatment.
      Part B only:
I 11. For patients who consent to paired biopsies (before treatment and during treatment): for baseline samples, formalin-fixed and paraffin-embedded (FFPE) archived biopsy samples can be used (within past 3 months prior to initiation of study treatment), but preferably fresh biopsies from primary tumor or recurrence or metastasis are collected. It is recommended that the end of cycle 2 on-treatment biopsy be collected at the same location as the baseline biopsy, whenever the tumor is accessible for a biopsy during treatment.

### Exclusion Criteria:

### General Exclusion Criteria Related to Study Methodology:

E 01. Eastern Cooperative Oncology Group (ECOG) performance status ≥2.
E 02. Significant concomitant illness, including psychiatric condition that, in the opinion of the Investigator or Sponsor, would adversely affect the patient's participation in the study.
E 03. Medical history or ongoing gastrointestinal disorders potentially affecting the absorption of compound (1). Patients unable to swallow normally and to take capsules. Predictable poor compliance to oral treatment.
E 04. Any malignancy related to human immunodeficiency virus (HIV); or unresolved viral hepatitis.
E 05. Patients with a life expectancy less than 3 months.
E 06. Patient not suitable for participation, whatever the reason, as judged by the Investigator, including medical or clinical conditions, or patients potentially at risk of noncompliance to the study procedures (i.e., unwillingness and inability to comply with scheduled visits, drug administration plan, laboratory tests, other study procedures, and study restriction).
E 07. Major surgery within 4 weeks prior to first study treatment administration.
E 08. Patient with any other cancer. However, adequately treated basal cell or squamous cell skin cancer or in situ cervical cancer or any other cancer from which the patient has been disease free for >3 years are allowed.
E 09. Patient is the Investigator or any subinvestigator, research assistant, pharmacist, study coordinator, or other staff or relative thereof directly involved in the conduct of the protocol.
   Part A only:
E 10. Patient with liver metastases only.

### Exclusion Criteria Related to the Disease:

E 11. Patients with known brain metastases, leptomeningeal carcinomatosis or/and spinal cord compression. Patients with brain metastases that have been previously totally resected or irradiated are eligible provided no progression or relapse is observed within 4 weeks of the treatment.
E 12. Treatment with anti-cancer agents (including investigational drugs) less than 2 weeks before first study treatment administration (less than 4 weeks if the anti-cancer agents were antibodies).
E 13. Prior treatment with another selective ER down-regulator (SERD) except fulvestrant for which a washout of at least 6 weeks is required prior to the first study drug administration.
E 14. Inadequate hematological function including neutrophils <1.5×10⁹/L; platelet count <100×10⁹/L.
E 15. Prothrombin time: International normalized ratio >1.5 times the upper limit of normal (ULN) or out of therapeutic range if receiving anticoagulation that would affect the PT/INR.
E 16. Inadequate renal function with serum creatinine ≥1.5×ULN or between 1.0 and 1.5×ULN with eGFR <60 mL/min/1.73 m² as estimated using the abbreviated Modification of Diet in Renal Disease formula, see below (with GFR designating the Glomerular Filtration rate and Src designating the serum creatinine):

GFR (mL/min/1.73 m²)=175×(Scr)−1.154×(Age)−0.203×(0.742 if Female)×(1.212 if African-American).
E 17. Liver function: aspartate aminotransferase (AST) >3×ULN (Upper Limit of Normal), or alanine aminotransferase (ALT) >3×ULN. Alkaline phosphatase (ALP) up to Grade 2 (2.5 to 5×ULN), gamma glutamyl transferase (GGT) up to Grade 2 (2.5 to 5×ULN) is acceptable only if related to the presence of bone and/or liver metastases as judged by the Investigator. Total bilirubin >1.5×ULN.
E 18. Patients with Gilbert disease.
E 19. Non-resolution of any prior treatment related toxicity to <Grade 2, except for alopecia according to National Cancer Institute Common Terminology Criteria for adverse events (NCI-CTCAE) v4.03.
E 20. Treatment with drugs that have the potential to inhibit UGT (including but not limited to atazanavir and probenecid), and treatment that are P-gp sensitive substrate (including but not limited to dabigatran, digoxin, and fexofenadine), less than 2 weeks before first study treatment administration or 5 elimination half-lives whichever is longest.
E 21. a. In all study parts: Treatment with strong and moderate CYP3A and CYP2C8 inducers within 2 weeks before first study treatment administration or 5 elimination half-lives whichever is longest (see list below).
   b. In parts A and B, in patients with 4β-OH cholesterol assessment: Treatment with strong and moderate CYP3A inhibitors within 2 weeks before first study treatment administration or 5 elimination half-lives whichever is longest.
E 22. More than 1 prior advanced CDK4/6 inhibitor based therapy.

### List of Strong and Moderate CYP3A4 Inducers:

The following tables are extracted from the January 2017 Drug-Drug Interaction Database from the University of Washington (www.druginteractioninfo.org).
In the tables below, AUC designates the area under the plasma concentration versus time.

STRONG CYP3A INHIBITORS

| | | | |
|---|---|---|---|
| | Precipitant Therapeutic | Victim (oral, unless otherwise | |
| CYP3A inhibitors | Class | specified) | AUC Ratio |

Potent CYP3A Inhibitors (yielding substrate AUCratio >5)

| | | | |
|---|---|---|---|
| Telaprevir | Antivirals | midazolam | 13.5 |
| Indinavir/RIT | Protease inhibitors | alfentanil | 36.50 |
| Tipranavir/RIT | Protease inhibitors | midazolam | 26.91 |
| Ritonavir | Protease inhibitors | midazolam | 26.41 |
| Cobicistat (GS-9350) | None | midazolam | 19.03 |
| Indinavir | Protease inhibitors | vardenafil | 16.25 |
| Ketoconazole | Antifungals | midazolam | 15.90 |
| Troleandomycin | Antibiotics | midazolam | 14.80 |
| Saquinavir/RIT | Protease inhibitors | midazolam | 12.48 |
| Itraconazole | Antifungals | midazolam | 10.80 |
| Voriconazole | Antifungals | midazolam | 9.63 |
| Mibefradil | Calcium Channel Blockers | midazolam | 8.86 |
| Clarithromycin | Antibiotics | midazolam | 8.39 |
| Danoprevir/RIT | Antivirals | midazolam | 13.42 |
| Lopinavir/RIT | Protease inhibitors | alfentanil | 11.47 |
| Elvitegravir/RIT | Treatments of AIDS | midazolam | 12.80 |
| Posaconazole | Antifungals | midazolam | 6.23 |
| Telithromycin | Antibiotics | midazolam | 6.2 |
| Grapefruit Juice DS | Food Products | midazolam | 5.95 |
| Conivaptan | Diuretics | midazolam | 5.76 |
| Nefazodone | Antidepressants | midazolam | 5.44 |
| Nelfinavir | Protease inhibitors | midazolam | 5.29 |
| Saquinavir | Protease inhibitors | midazolam | 5.18 |
| Boceprevir | Antivirals | midazolam | 5.05 |
| Idelalisib | Kinase inhibitors | midazolam | 5.15 |
| LCL 161 | Cancer treatments | midazolam | 8.80 |
| VIEKIRA PAK | Antivirals | tacrolimus | 55.76 |

MODERATE CYP3A INHIBITORS

| | | | |
|---|---|---|---|
| | Precipitant Therapeutic | Victim (oral, unless otherwise | |
| CYP3A inhibitors | Class | specified) | AUC Ratio |

Moderate CYP3A Inhibitors (AUCratio ≥2 and <5)

| | | | |
|---|---|---|---|
| Erythromycin | Antibiotics | midazolam | 4.99 |
| Fluconazole | Antifungals | midazolam | 4.93 |
| Atazanavir/RIT | Protease inhibitors | maraviroc | 4.90 |
| Darunavir | Protease inhibitors | saquinavir | 4.90 |
| Diltiazem | Calcium Channel Blockers | midazolam | 4.06 |
| Darunavir/RIT | Protease inhibitors | sildenafil | 4.00 |
| Dronedarone | Antiarrhythmics | simvastatin | 3.66 |
| Crizotinib | Kinase Inhibitors | midazolam | 3.65 |
| atazanavir | Protease Inhibitors | maraviroc | 3.57 |
| GSK2647544 | Alzheimer's Disease & | simvastatin | 3.30 |
| | Dementia | | |
| aprepitant | Antiemetics | midazolam | 3.29 |
| casopitant | Antiemetics | midazolam | 3.13 |
| amprenavir | Protease Inhibitors | rifabutin | 2.93 |
| faldaprevir | Antivirals | midazolam | 2.92 |
| imatinib | Antineoplastic Agents | simvastatin | 2.92 |
| verapamil | Calcium Channel Blockers | midazolam | 2.92 |
| netupitant | Antiemetics | midazolam | 2.44 |
| nilotinib | Kinase Inhibitors | midazolam | 2.40 |
| grapefruit juice | Food Products | midazolam | 2.39 |
| tofisopam | Benzodiazepines | midazolam | 2.36 |
| cyclosporine | Immunosuppressants | midazolam | 2.21 |
| ACT-178882 | Renin Inhibitors | midazolam | 2.19 |
| ciprofloxacin | Antibiotics | sildenafil | 2.12 |
| schisandra | Herbal Medications | midazolam | 2.05 |
| sphenanthera | | | |
| isavuconazole | Antifungals | midazolam | 2.03 |
| cimetidine | H-2 Receptor Antagonists | midazolam | 2.02 |
| FK1706 | Central Nervous System | midazolam | 2.01 |
| | Agents | | |

The following tables are extracted from the April 2019 Drug-Drug Interaction Database from the University of Washington (www.druginteractioninfo.org).

STRONG CYP3A INDUCERS

| | | | | | |
|---|---|---|---|---|---|
| | | Victim (oral | % of | % oral | |
| | Therapeutic | unless otherwise | AUC | CL | Precipitant |
| Inducers | Class | specified) | decrease | increase | dose (oral) |

Potent Inducers of CYP3A (AUC decreased by ≥80% or CL increased by more than 5-fold [400%])

| | | | | | |
|---|---|---|---|---|---|
| Rifampin | Antibiotics | Budesoinide | 99.7 | 36904.5 | 600 mg QD (7 days) |
| Mitotane | Other | Midazolam | 94.5 | Not provided | Maximum of 3.5 g TID |
| | Antineoplastics | | | | (chronic therapy) |
| Avasimibe | Other | Midazolam | 93.5 | Not provided | 750 mg/day (7 days) |
| | Antilipemics | | | | |
| Phenytoin | Anticonvulsants | Nisoldipine | 89.5 | Not provided | 200-450 mg/day |
| | | | | | (chronic treatment) |
| Carbamazepine | Anticonvulsants | Quetiapine | 86.6 | 643.1 | 200 mg TID (26 days) |
| Enzalutamide | Antiandrogens | Midazolam | 85.9 | Not provided | 160 mg QD (85 ± 3 days) |
| St John's Wort | Herbal | Midazolam | 80.0 | Not provided | 300 mg TID 14 days |
| extract | Medicines | | | | |
| Rifabutin | Antibiotics | Delavirdine | Not | 458.0 | 300 mg QD 14 days |
| | | | provided | | |
| Phenobarbital | Anticonvulsants | Verapamil | 76.6 | 400.9 | 100 mg QD 21 days |

MODERATE CYP3A INDUCERS

| | | | | | |
|---|---|---|---|---|---|
| | | Victim (oral | % of | % oral | |
| | Therapeutic | unless otherwise | AUC | CL | Precipitant |
| Inducers | Class | specified) | decrease | increase | dose (oral) |

Moderate inducers of CYP3A (AUC decreased by 50-80% or CL increased by 2 to 5-fold [100%-400%])

| | | | | | |
|---|---|---|---|---|---|
| Ritonavir and | None | Midazolam | 77.2 | Not | Ritanovir 300 mg BID; St John's |
| St John's Wort | | | | provided | Wort 300 mg TID (14 days) |
| Semagacestat | Alzheimer's | Midazolam | 76.4 | 324.6 | 140 mg QD (10 days) |
| | Treatments | | | | |
| Tipranavir and | Protease | Saquinavir | 75.6 | Not | Tipranavir 500 mg; ritonavir 200 |
| ritonovir | inhibitors | | | provided | mg BID (14 days) |
| Bosentan | Endothelin | Sildenafil | 69 | 239.8 | 62.5-125 mg BID 8 weeks |
| | receptor | | | | |
| | antagonist | | | | |
| Genistein | Food Products | Midazolam | 13.7 | 136.9 | 1000 mg QD (14 days) |
| hioridazine | Antipsychotics | Quetiapine | 68.7 | 104.5 | 100-300 mg QD (15 days) |
| Naficillin | Antibiotics | Nifedipine | 62.6 | 145.1 | 500 mg 4 times daily 5 days |
| Talviraline | NNRTIs | Indinavir | 61.7 | 181.2 | 500 mg TID 14 days |
| Efavirenz | NNRTIs | Simvastatin acid | 60.4 | Not | 600 mg QD 28 days |
| | | | | provided | |

MODERATE CYP3A INDUCERS

| | | | | | |
|---|---|---|---|---|---|
| | | Victim (oral | | | |
| | | unless | % of | % oral | |
| | Therapeutic | otherwise | AUC | CL | Precipitant |
| Inducers | Class | specified) | decrease | increase | dose (oral) |

Moderate inducers of CYP3A (AUC decreased by 50-80% or CL increased by 2 to 5-fold [100%-400%])

| | | | | | |
|---|---|---|---|---|---|
| Lopinavir | Protease | Amprenavir | 59.7 | Not | 400 mg BID (4 weeks) |
| | inhibitors | | | provided | |
| Modafinil | Psycho- | Triazolam | 57.6 | 35.7 | 200-400 mg QD 28 days |
| | stimulants | | | | |
| Etravirine | NNRTIs | Sildenafil | 56.7 | Not | 800 mg BID 13.5 days |
| | | | | provided | |
| Lersivirine | NNRTIs | Midazolam | 51.4 | 105.5 | 1000 mg BID (14 days) |

The following tables are extracted from the April 2019 Drug-Drug Interaction Database from the University of Washington (www.druginteractioninfo.org). Strong CYP2C8 inducers are not listed in the table below as there are none identified currently.

MODERATE CYP2C8 INDUCERS

| | | | | |
|---|---|---|---|---|
| | | | Max % of AUC | |
| CYP2C8 | Therapeutic | Inducer | decrease or CL | substrate |
| inducers | Class | dose (oral) | increase | (oral) |

Moderate Inducers (50% ≤ AUC decrease <80% or 100% ≤ CL increase <400%)

| | | | | |
|---|---|---|---|---|
| | | 600 mg QD (7 days) | 79.6 | repaglinide |
| rifampin | Antibiotics | 600 mg QD (6 days) | 66.4 | rosiglitazone |
| | | 600 mg QD (6 days) | 53.8 | pioglitazone |
| carbamazepine | Anticonvulsants | 200 mg QD (3 days) then | 70.3 | dasabuvir |
| | | BID (21 days) | | |
| ivosidenib | Cancer | 1200 mg QD (19 days) | 59.0 (PBPK) | repaglinide |
| | Treatments | | | |
| hormonal | Oral | ethinyl | 54.5 | dasabuvir |
| contraceptives | Contraceptives | estradiol/norgestimate | | |
| | | 35/250 μg QD (21 days) | | |

Medications with a Narrow Therapeutic Window Metabolized by CYP3A:

| CYP enzyme | NTR (Narrow Therapeutic Range) Substrates |
|---|---|
| CYP3A | Alfentanil, cyclosporine, dihydroergotamine, |
| | ergotamine, fentanyl, quinidine, sirolimus, |
| | tacrolimus,cisapride, astemizole, |
| | terfenadine, pimozide |

CYP Substrates with a Narrow Therapeutic Range - drugs with an exposure-response relationship that indicates that relatively small increases in their exposure levels by coadministered CYP inhibitors may lead to safety concerns.

Patients treated or intended to be treated with the following drugs metabolized by CYP3A/CYP2B6/CYP2C and/or UGT (Uridine Glucuronosyltransferase) should be carefully monitored for their efficacy, since their exposure may be decreased by compound (1):

| Drug | Therapeutic Class |
|---|---|
| Adinazolam | Anxiolytic |
| Aliskiren | Antihypertensive |
| Alprazolam | Anticonvulsant~Anxiolytic~Hypnotic~Myorelaxant~Sedative |
| Buspirone | Anxiolytic |
| Casopitant | Antiemetic |
| Conivaptan | Diuretic |
| Darifenacin | Antidiuretic |
| Diltiazem | Antianginal~Antihypertensive~Calcium channel blocker~Vasodilator |
| Dronedarone | Antiarrhytmic |
| Ebastine | Antihistaminic |
| Gepirone | Antidepressant~Anxiolytic |
| Ketamine | General Anaesthesic |
| Lopinavir | Antiviral |
| Lovastatin | Anticholesterol |
| Midazolam | Anaesthesic~General Anaesthesic~Hypnotic~Sedative |
| Nisoldipine | Antihypertensive~Calcium channel blocker |
| RWJ 51204 | Anticonvulsant~Anxiolytic |
| Rapamycin | Antineoplastic: Immunosuppressant~Immunosuppressant |
| Simvastatin | Anticholesterol |
| Testosterone | Androgen |
| Triazolam | Anticonvulsant~Anxiolytic~Hypnotic~Myorelaxant~Sedative |
| Vardenafil | Erectil Anti-dysfunction~Vasodilator |
| alpha-Endosulfan | |

1-4: Study Treatment

Compound (1) is administered by the oral route and in the form of 10, 50 and 100 mg capsules, stored between 2° C. and 8° C.

In Part A, Compound (1) is administered at assigned dose levels, within a 28-day cycle. For the QD regimen, during Cycle 1, one dose is taken on Day 1 in fasting condition, no dose on Day 2 and repeated administration starts from Day 3. On Day 3 only, the dose is taken in fed condition for a food effect evaluation, and then all subsequent administrations are taken in fasted condition, and at approximately the same time each day (±3 hours). From the conclusions drawn with the initial QD dose regimen, patients treated with other dose regimens are allowed to take Compound (1) in fasted or fed condition. For the BID regimen, the 600 mg dose is split in two drug administrations: 300 mg taken two times a day 12 hours apart (i.e. 2×300 mg)±1 hour with or without food (with one dose only taken in the morning on Day 1, no dose on Day 2 and then repeated BID dosing from Day 3).

In Part B, Compound (1) is taken QD from Day 1 (without omission on Day 2) at the RD fixed in Part A, within a 28-day cycle, either in a fasting or fed condition; and at approximately the same time each day (±3 hours). A BID regimen (without omission on Day 2) may also be explored if found to be needed.

If a dose is vomited or omitted, the patient should not take the dose later or 2 doses at the next planned dose, and this information has to be recorded in the diary.

Patients are advised to avoid sun exposure and wear protective clothing, sunscreen, and lip balm with high sun protection (eg, factor 30) when outdoors during study treatment and until 5 days after the last administration of compound (1).

Concomitant Treatment:

A concomitant medication is any treatment received by the patient concomitantly to any study treatment(s). All treatments being taken by the patient from the date of the consent form to the first study treatment administration, at any time during the treatment period and up to 30 days after the last dose are regarded as prior and concomitant treatments respectively, and shall be reported on the appropriate screen of the eCRF.

Premenopausal patients treated with a GnRH analog for at least 6 months to be eligible before study treatment initiation must continue this GnRH analog during study treatment period.

Drugs which are mainly metabolized by CYP3A, CYP2B6, CYP2Cs and/or UGT shall be closely monitored since the efficacy of these drugs may be decreased by concomitant use of compound (1) due to a potential induction effect of said compound at higher dose planned in this study. Special caution should be taken with regards to the following therapies:
Proton Pump Inhibitors (PPI) (i.e., omeprazole): when prescribed, compound (1) intake should be preferred with food.
Drugs that are contra-indicated with UGT substrates, including but not limited to atazanavir and probenecid, since compound (1) is substrate of UGT1A1 and UGT1A4.
The concomitant use of drugs for which the approved label indicates contraindication or precaution with CYP3A potent and/or moderate inducers since compound (1) may decrease their exposure.

While participating in this study, patients may not receive any standard or investigational agents for treatment of their tumor other than the compound (1). Any medication which is considered necessary for the patient's welfare, and which is not expected to interfere with the evaluation of the study drug, may be given at the discretion of the Investigator, with the exception of those listed below.

Prohibited in Parts A and B:

Systemic non-authorized anti-cancer agents or concomitant radiotherapy (localized radiation therapy for pain control is permitted without any change in the study treatment).

Among herbal medications and food products, it is recommended to avoid consumption of St John's Wort and genistein during treatment period

Patients should avoid direct exposure to natural or artificial sunlight during the study treatment period.

The following therapies since they may increase compound (1) exposure by more than 2-fold: antiviral agent (atazanavir, lopinavir), antifungal (ketoconazole), antioxidant (quercetin), strong and moderate CYP3A4 and CYP2C8 inducers (see list above). Drugs that are substrate of P-gp should be avoided (dabigatran, digoxin, fexofenadine).

In addition, for the 12 patients with full PK sampling to be included in the study: initiation of any treatment with known strong and moderate CYP3A inhibitors is not allowed 2 weeks before initiation and during Cycle 1. These treatments would interfere with 4β-hydroxy/total cholesterol test interpretation.

Patients taking any of the above prohibited medications at the time of the screening visit are ineligible to enter study until administration of the prohibited agent can be safely discontinued, and an appropriate period of time has elapsed (2 weeks before first study treatment administration or 5 elimination half-lives, whichever is longest).

If a patient's clinical status requires administration of any of the above prohibited medications during the study, administration of the study drug shall be stopped, and the patient shall be withdrawn from the study. The change in clinical status mandating the use of the medication in question must be reported as the reason for study drug discontinuation.

1-5: Endpoints

### 1-5-1: Primary Endpoint

### Part A:

Incidence of study treatment-related DLTs at Cycle 1 (Day 1 to Day 28). DLT is defined as the occurrence of any of the following treatment-emergent adverse events (TEAEs) related to the study therapy using NCI-CTCAE (v4.03):
Any Grade ≥3 non-hematological toxicity, except:
Grade 3 nausea and vomiting resolving to Grade ≤1 within 48 hours, with or without adequate antiemetic treatment,
Grade 3 diarrhea, if controlled with adequate antidiarrheal therapy and lasting less than 48 hours.
Any Grade ≥3 hematological toxicity, except
Grade 3 anemia,
Grade 4 neutropenia <7 days,
Grade 3 neutropenia without fever or infection,
Grade 3 thrombocytopenia without bleeding.
Any elevated total serum bilirubin >2×ULN.
Any toxicity related to study treatment, resulting in omission of the study treatment for 7 days or more during Cycle 1.
A TEAE that in the opinion of the study committee is possibly or probably study treatment-related and is of potential clinical significance such that further dose escalation would expose patients to unacceptable risk.

These TEAEs are considered as study treatment-related in the absence of evidence to the contrary and if not related to disease progression. If multiple toxicities are seen, the presence of DLT will be based on the most severe experienced toxicity. At the end of Cycle 1, each patient must be assessed by the Investigator as to whether or not the patient experienced DLT, and this information must be recorded within the appropriate screen of the electronic case report forms (eCRFs) and an electronic DLT notification (either DLT or not) is sent to the Sponsor. Patients can continue the treatment after resolution (Grade 1) of the adverse event (AE) or to their baseline status. Patients need to complete at least 75% of the intended doses at Cycle 1 to be evaluable and shall have ¹⁸FES-PET scans evaluable at baseline and between Day 11 and Day 15 to be evaluable for DLTs in Part A.

DLT-nonevaluable patients are replaced, and additional patients are enrolled if needed for dose-escalation decisions after agreement within the study committee.

### Part B:

ORR: antitumor activity as documented by tumor response (CR or PR) defined by RECIST v1.1 determined by Independent Central Review (ICR).

### 1-5-2: Secondary Endpoints

Overall safety profile of compound (1), and characterization in terms of the type, frequency, severity, and relationship to study therapy of any AEs or abnormalities of physical findings, laboratory tests, or electrocardiograms (ECGs); drug discontinuation/omission/reduction and cycle delay due to AEs; or serious adverse events (SAEs).
ORR as per RECIST v1.1 assessed by investigators/local radiologists.
ORR and CBR in patients based on their ESR1 status (mutated or wild type) analyzed by multiplex droplet digital polymerase chain reaction (ddPCR) after extraction of plasma circulating DNA (Part B). ORR assessed by investigators/local radiologists, and in Part B also determined by ICR.
Clinical benefit (CR+PR+SD≥24 weeks) as per RECIST v1.1 assessed by investigators/local radiologists, and in Part B also determined by ICR.
Duration of response assessed by investigators/local radiologists, and in Part B also determined by ICR.
Non-progression rate at 6 months (percentage of patients without progression at 6 months) assessed by investigators/local radiologists, and in Part B also determined by ICR.
Time to first tumor response (CR or PR) in Part B is defined as the time interval from the date of first administration of the study treatment to the date of the first occurrence of CR or PR, assessed by investigators/local radiologists and in Part B also determined by ICR.
To correlate the changes observed in FES-PET scan with the changes in glucose metabolism seen on FDG PET/CT in Part A.
PK parameters for compound (1) during Cycle 1:
After a single dose (on Day 1 Part A fasted state and in Part B, and Day 3 in Part A fed state): at least t_{lag} (lag time, interval between administration time and the sampling time preceding the first concentration above the lower limit of quantification), tₘₐₓ (first time to reach Cₘₐₓ), Cₘₐₓ (maximum concentration observed), AUC₀₋₂₄ (area under the plasma concentration versus time curve from time zero to 24 hours) or AUC₀₋₁₂ and if possible for Part A Day 1, AUC, t_{1/2z} (terminal half-life associated with the terminal slope (λz)) and CL/F (apparent total body clearance of the drug from the plasma).
After repeated administration on Day 22: at least tₘₐₓ, Cₘₐₓ, AUC₀₋₂₄ or AUC₀₋₁₂.
C_{trough} (plasma concentration observed before treatment administration) at T=0 h, i.e. before daily administration, is also obtained over Cycle 1.
Urine excretion of compound (1) is estimated during the expansion phase (Part B).
Potential for CYP3A enzyme induction and inhibition by compound (1): plasma 4β-hydroxy/total cholesterol concentration ratios on Day 22 versus Day 1 (Part B) or Cycle 2 Day 1 versus Day 1 (Part A).
Inhibition of ER occupancy at ¹⁸FES-PET imaging (signal extinction). All patients in Part A have an ¹⁸FES-PET/CT scan imaging performed at baseline and on treatment in addition to an FDG PET/CT. For patients on a QD regimen, the second scan is performed after at least 8 continuous days of treatment (i.e., between Day 11 and Day 15) and between 16 to 24 hours after the previous administration of the study drug, with a time window of 2 hours around 24 hour theoretical time. For patients on a BID regimen, the second scan for both FES-PET and FDG PET/CT is performed after at least 8 days of continuous treatment (i.e., between Day 11 and Day 15 post first study treatment dose) and between 7 to 12 hours after the previous administration of the drug. The signal extinction between baseline and on study treatment ¹⁸FES-PET scans constitute the PD readout of the ER engagement. No ¹⁸FES-PET or FDG PET/CT imaging is performed in Part B.

1-5-3: Exploratory Endpoints

ER Degradation

Patients who consented to paired biopsies are asked to contribute the most recent archived biopsy (within past 3 months prior to initiation of study treatment) or preferably to a fresh tumors biopsy at baseline and at the end of Cycle 2. A total of 9 FFPE slides (5 micron each for IHC analysis) and 3 FFPE slides (10 μm each for RNA analysis, if possible) are collected for each biopsy. The presence of ER is determined by central IHC and the ER results at baseline and on treatment are compared to assess ER degradation.

ESR1 Mutation Status

Twelve independent mutations of ESR1 gene are determined in all patients at baseline and at end of Cycle 2 (Day 15 to Day 28) by ddPCR from plasma extracted cfDNA.

The clinical responses are also assessed in the ESR1 wild type and the ESR1 mutated population separately in Part B.

Mutational Profile

In all patients, plasma is collected at baseline and at the EOT and cfDNA is extracted. The mutation status of a limited number of cancer genes is determined by next generation sequencing and the potential link between specific mutation and clinical outcomes is investigated to understand intrinsic or acquired resistance to the compound (1) treatment.
To evaluate PK/PD relationships of compound (1) with ER occupancy, PD and/or efficacy endpoints and/or other breast cancer biomarkers such as Ki67, Bcl-2 and PgR.
To evaluate FDG SUV uptake before and during treatment with compound (1) (Part A).
To correlate the changes observed in FES-PET scan with the changes in glucose metabolism seen on FDG PET/CT in Part A.

1-6: Statistical Considerations

Sample Size Justification:

The actual sample size can vary depending on DLTs observed, number of dose levels actually explored and the other potential schedules to be tested.

According to different simulated scenarios, it is anticipated that approximately 25 DLT-evaluable patients shall enter into the monotherapy dose escalation phase (Part A) of the study in QD regimen and 12 DLT-evaluable patients into the monotherapy dose escalation phase (Part A) in BID regimen.

For Part B, it is anticipated that approximately 78 patients shall enter into the dose expansion cohort of the study. If the BID regimen is tested in Part B, based on any relevant information from data in patients treated with the BID regimen in Part A and patients treated with the QD regimen in Part B, it is anticipated that 56 additional patients shall enter into the dose expansion cohort (Part B) of the study in the BID schedule.

General Statistical Approach:

DLT-evaluable population in dose escalation phase includes all patients who have received a first complete cycle (28-day, oral administration), taking at least 75% of the intended dosing, unless the patient discontinued the study treatment before Cycle 1 completion for a DLT and in Part A have an evaluable ¹⁸FES-PET scans at baseline and between Day 11 and Day 15 of the first cycle. Any patient who develops a DLT in Part A despite the absence of evaluable ¹⁸FES-PET scan are included in the DLT population. A patient not evaluable who discontinues the study treatment before the end of Cycle 1 for any reason other than DLT shall be replaced.

Safety population includes all patients exposed to at least one dose of the study treatment.

Response evaluable patients are defined as treated patients with measurable disease at study entry who have at least one postbaseline evaluable tumor assessment. Patients with an early progression as per RECIST v1.1 or who died from disease progression are evaluable for response.

PK evaluation is performed on all patients without any major deviations related to study treatment administration (eg, early vomiting just after drug administration), and for whom any PK parameters are available.

Part A (Dose Escalation Monotherapy):

Study treatment-related DLTs occurring during Cycle 1 are assessed and analyzed on the DLT-evaluable population. In addition, AEs meeting DLT criteria occurring in any additional cycle are assessed and analyzed on the safety population.

Safety and PK data are descriptively summarized for each dose level on the safety population.

Treatment emergent adverse events (TEAEs) are analyzed according to current version of the Medical Dictionary for Regulatory Activities (MedDRA) dictionary. Laboratory abnormalities are analyzed according to the NCI-CTCAE v.4.03. Type, frequency, seriousness, severity and relatedness of study treatment TEAEs is analyzed on the safety population for each dose level.

Dose proportionality is assessed on pooled data from Parts A and B, using a power model on Cₘₐₓ, and AUC₀₋₂₄ on Day 1 and Day 22.

Accumulation ratio (Day 22/Day 1) for Cₘₐₓ and AUC₀₋₂₄ is estimated with 90% confidence intervals (CI) using a linear fixed effects model on log transformed parameters.

Within-subject and total standard deviations for log(Cₘₐₓ) and log(AUC₀₋₂₄) are estimated (Parts A and B).

The food effect is assessed by comparing AUC₀₋₂₄ and Cₘₐₓ between Day 1/Cycle 1 and Day 3/Cycle 1 in Part A.

4β-hydroxy/total cholesterol concentration ratio (Cycle2/Cycle1) is estimated with 90% CI.

Preliminary efficacy is descriptively presented on activity/efficacy population.

Part B (Dose Expansion Monotherapy):

For the study design, it is assumed that the response rate is 10% under the null hypothesis according to literature with fulvestrant monotherapy in this population. It is expected that compound (1) would induce a response rate of 20%. Under these hypotheses, with a one-sided 5% significance level with a power of 80%, it is anticipated that 78 patients for efficacy are to be entered into the dose expansion cohort of the study.

An interim analysis of the response rate shall be done after treatment of the first 45 response patients. It is expected to have at least 5 patients with response (CR or PR) in order to continue the enrolment up to 78 patients; other parameters are also to be taken into consideration such as the duration of response, the CBR (CR+PR+SD≥24 weeks), the percentage of patients with SD and the duration of SD.

If a BID regimen is tested in the expansion cohort, with a one-sided 10% significance level with a power of 80%, it is anticipated that 56 patients are to be entered into the dose expansion cohort of the study.

Similar analyses as for the escalation phase shall be performed.

Response rate and other clinical outcomes are also to be presented according to ESR1 status (mutation or wild type) for interim and final analysis.

4β-hydroxycholesterol concentration ratio (Day 22/Day 1) is estimated with 90% CI.

1-7: Duration of Study

Duration of Entire Study Per Patient:

The duration of the study for an individual patient includes a period to assess eligibility (screening period) of up to 4 weeks (28 days), a treatment period of at least 1 cycle (28 days) of study treatment, and an EOT visit at least 22 to 30 days (or until the patient receives another anti-cancer therapy, whichever is earlier) following the last administration of study treatment. If further therapy is initiated before day 22 after last IMP (Investigational Medicinal Product), investigator should contact the patient (either by phone call or visit) to obtain final collection of safety information (stabilization or recovery of TEAEs) within 30 days after last IMP intake or in follow up visit in case of ongoing SAE or related AE. Study treatment may continue until precluded by unacceptable toxicity, disease progression, or upon patient's request. Patients who discontinue the study treatment prior to documentation of PD are followed-up every 2 months until disease progression or initiation of further anti-cancer therapy, or cut-off date (COD), whichever comes first. Treatment may stop at any time if the study is terminated by the Sponsor.

Duration of Entire Study:

The expected enrolment period is approximately 30 months. The first 2 trial CODs shall be at the end of the first cycle of the last patient treated in the escalation phase (Part A) in order to have at least the first cycle evaluable for all patients for determination of the MTD and for the RD.

The other 2 CODs shall be when the last patients treated in the expansion cohorts (Part B) have 2 tumor assessments or early progression or EOT tumor assessment, whichever occurs first, in order to assess tumor response. After the last COD, ongoing patients shall receive study therapy until disease progression, occurrence of an AE leading to treatment discontinuation, whichever is earlier, and they shall only be followed for study treatment administration, SAEs, study treatment-related AEs and AEs leading to study treatment discontinuation.

The end of study occurs when all patients have had the opportunity to complete the EOT visit 30 days after the last study treatment administration.

1-8: ¹⁸FES-PET/CT Assessments

Molecular imaging is a useful tool for measuring drug effects in cancer patients. ¹⁸FES-PET/computerized tomography (CT) has been validated as an accurate method for localizing ER-expressing tumors and as a predictive assay for breast cancer endocrine therapy. In breast cancer, the uptake of ¹⁸FES, as measured by standardized uptake value (SUV) on PET, has been shown to correlate with ER expression in biopsy material assayed by in vitro radioligand binding and by immunohistochemistry (IHC), providing evidence of the value of ¹⁸FES SUV to measure specific binding to ER.

Inhibition of ER occupancy investigation using ¹⁸FES-PET imaging is hence a limited invasive procedure that allows to assess ER presence by assessing the binding of radiolabelled estradiol, the ligand of ER (signal extinction). ER-positive tumor sites are detected with ¹⁸FES-PET/CT scan imaging at baseline and are compared to a scan evaluated at steady state. For a patient on a QD regimen, the second scan is performed after at least 8 continuous days of treatment (i.e., between Day 11 and Day 15) and between 16 to 24 hours after the previous administration of the study drug, with a time window of 2 hours around 24 hour theoretical time. For patients on a BID regimen, the second scan is performed after at least 8 continuous days of treatment (i.e., between Day 11 and Day 15) and between 7 to 12 hours after the previous administration of the study drug. In case of limited response observed by PK and ¹⁸FES-PET, a twice daily schedule of administration is considered for the RD decision after using ¹⁸FES-PET at an earlier time window. Patients should be instructed to take the previous study treatment dose at the appropriate time in line with availability of the PET/CT scan at sites. The signal extinction between baseline and on study treatment ¹⁸FES-PET scans (ΔSUV) constitutes the PD readout of the ER engagement. A decrease ≥90% inhibition of SUV should represent an inhibition of the estrogen ligand binding or the near-to complete degradation of ER. The ¹⁸FES-PET follows the extent of ER inhibition and this helps with the RD decision. No ¹⁸FES-PET imaging is performed in Part B.

Study results presented below stem from a cut-off date at database extraction on 31 Mar. 2020.

2: Part A Results and Patients Characteristics in Parts A and B of the Study

In the dose-escalation part of compound (1) single agent (Part A), a total of 16 patients have been treated. Five dose levels of compound (1) QD have been explored: 20 mg (3 patients), 150 mg (3 patients), 200 mg (4 patients), 400 mg (3 patients), and 600 mg (3 patients).

In part A the mean age was 59.5 (±10.9) years, the 400 mg cohort had the oldest mean age (mean 68.3 years), and the 150 mg cohort had the youngest mean age (mean 54.3 years). Eastern Cooperative Oncology Group (ECOG) status was 1 in 37.5% of the patients and ECOG 0 in the remaining patients. The median time from first diagnosis to first study treatment administration was 9.7 years: the shortest time was 2.3 years at 200 mg dose level and the longest was 22.7 years at 150 mg dose level.

All patients were ER− positive and HER2− negative. 81.3% of the patients were progesterone receptor positive.

All patients were metastatic. They had a median of 2 organs involved at study entry (range 2 to 4) in Part A; the main organs involved (≥50%) are lymph nodes, bone, and liver.

Patients were heavily pretreated, receiving several lines of anti-cancer treatments, including chemotherapy, hormonal therapy and targeted therapy, with a median of 2.5 prior anti-cancer therapies (range 1-8) in the advanced setting; 93.8% received prior aromatase inhibitor, 75.0% were treated with a CDK 4/6 inhibitor, 56.3% received prior SERD-based therapy, 43.8% mTOR inhibitor and 31.3% SERM.

The demographic and the baseline disease characteristics of the patients treated in Part A and Part B of TED14856 study are provided in Table 1a and 1b. In those tables, “pooled population” designates the Part A patients receiving compound (1) ≥150 mg QD (excluding 3 patients treated at the 20 mg QD dose) and the Part B patients.

TABLE 1a
TED14856 - Demographic and patient characteristics
at baseline in Part A and B

| | | |
|---|---|---|
| | Compound (1) | |
| Compound (1) | 400 mg | Pooled |
| PART A | PART B | Population |
| (N = 16) | (N = 49) | (N = 62) |

Age (years)

| Number | 16 | | 49 | | 62 | |
|---|---|---|---|---|---|---|
| Mean (SD) | 59.5 | (10.9) | 63.1 | (10.6) | 62.2 | (10.8) |

| | | | |
|---|---|---|---|
| Median | 59.5 | 63.0 | 63.0 |
| Min; Max | 40; 79 | 37; 88 | 37; 88 |

Age group (years) [n(%)]

| Number | 16 | | 49 | | 62 | |
|---|---|---|---|---|---|---|
| [18-64] | 11 | (68.8) | 29 | (59.2) | 39 | (62.9) |
| [65-84] | 5 | (31.3) | 17 | (34.7) | 20 | (32.3) |

| | | | | | |
|---|---|---|---|---|---|
| ≥85 | 0 | 3 | (6.1) | 3 | (4.8) |

Race [n(%)]

| Number | 10 | | 36 | | 45 | |
|---|---|---|---|---|---|---|
| White | 10 | (100) | 35 | (97.2) | 44 | (97.8) |

| | | | | | |
|---|---|---|---|---|---|
| Asian | 0 | 1 | (2.8) | 1 | (2.2) |

Ethnicity [n(%)]

| Number | 16 | | 49 | | 62 | |
|---|---|---|---|---|---|---|
| Hispanic or Latino | 0 | | 3 | (6.1) | 3 | (4.8) |
| Not Hispanic or Latino | 8 | (50.0) | 28 | (57.1) | 36 | (58.1) |
| Unknown | 1 | (6.3) | 0 | | 1 | (1.6) |
| Not reported | 7 | (43.8) | 18 | (36.7) | 22 | (35.5) |

Baseline Weight (kg)

| Number | 16 | | 49 | | 62 | |
|---|---|---|---|---|---|---|
| Mean (SD) | 72.67 | (12.76) | 71.95 | (16.83) | 72.01 | (16.21) |

| | | | |
|---|---|---|---|
| Median | 74.05 | 70.50 | 71.25 |
| Min; Max | 50.7; 105.5 | 41.0; 115.8 | 41.0; 115.8 |

EGOC PS [n(%)]

| Number | 16 | | 49 | | 62 | |
|---|---|---|---|---|---|---|
| 0 | 10 | (62.5) | 29 | (59.2) | 37 | (59.7) |
| 1 | 6 | (37.5) | 20 | (40.8) | 25 | (40.3) |

Time from first diagnosis to first
study treatment administration (years)

| Number | 16 | | 45 | | 58 | |
|---|---|---|---|---|---|---|
| Mean (SD) | 10.73 | (7.04) | 7.82 | (6.17) | 8.29 | (6.29) |

| | | | |
|---|---|---|---|
| Median | 9.67 | 6.04 | 6.70 |
| Min; Max | 2.3; 22.7 | 0.8; 24.3 | 0.8; 24.3 |

Histology type [n(%)]

| Number | 16 | | 49 | | 62 | |
|---|---|---|---|---|---|---|
| Adenocarcinoma | 13 | (81.3) | 35 | (71.4) | 45 | (72.6) |
| Other | 3 | (18.8) | 14 | (28.6) | 17 | (27.4) |

Staging [n(%)]

| Number | 16 | | 49 | | 62 | |
|---|---|---|---|---|---|---|
| Stage 0 | 0 | | 2 | (4.1) | 2 | (3.2) |
| Stage I | 3 | (18.8) | 3 | (6.1) | 6 | (9.7) |
| Stage II | 4 | (25.0) | 13 | (26.5) | 17 | (27.4) |
| Stage III | 1 | (6.3) | 7 | (14.3) | 8 | (12.9) |
| Stage IV | 5 | (31.3) | 16 | (32.7) | 20 | (32.3) |
| Unknown | 3 | (18.8) | 8 | (16.3) | 9 | (14.5) |

HER2 Status

| Number | 15 | | 49 | | 61 | |
|---|---|---|---|---|---|---|
| Negative | 15 | (100) | 49 | (100) | 61 | (100) |

ER Status

| Number | 16 | | 49 | | 62 | |
|---|---|---|---|---|---|---|
| Positive | 16 | (100) | 49 | (100) | 62 | (100) |

PR (progesterone receptor) Status

| Number | 16 | | 47 | | 60 | |
|---|---|---|---|---|---|---|
| Negative | 3 | (18.8) | 13 | (27.7) | 16 | (26.7) |
| Positive | 13 | (81.3) | 34 | (72.3) | 44 | (73.3) |

Number of organ(s) involved

| Number | 16 | | 49 | | 62 | |
|---|---|---|---|---|---|---|
| Mean (SD) | 2.63 | (0.81) | 2.73 | (1.20) | 2.71 | (1.14) |

| | | | |
|---|---|---|---|
| Median | 2.00 | 3.00 | 2.50 |
| Min; Max | 2.0; 4.0 | 1.0; 6.0 | 1.0; 6.0 |

Main Type of organ(s) involved

| | | | | | | |
|---|---|---|---|---|---|---|
| Bone | 11 | (68.8) | 35 | (71.4) | 45 | (72.6) |
| Breast | 1 | (6.3) | 12 | (24.5) | 13 | (21.0) |
| Liver | 11 | (68.8) | 28 | (57.1) | 37 | (59.7) |
| Lung | 5 | (31.3) | 15 | (30.6) | 19 | (30.6) |
| Lymph node | 9 | (56.3) | 19 | (38.8) | 26 | (41.9) |
| Pleura | 1 | (6.3) | 10 | (20.4) | 11 | (17.7) |

TABLE 1b
TED14856 - Demographic data: prior anti-cancer treatments Part A and B

| | | |
|---|---|---|
| | Compound (1) | |
| Compound (1) | 400 mg | Pooled |
| PART A | PART B | Population |
| (N = 16) | (N = 49) | (N = 62) |

Type of prior anti-cancer treatment
(advanced or metastatic disease)

| Prior chemotherapy | 8 | (50.0) | 20 | (40.8) | 26 | (41.9) |
|---|---|---|---|---|---|---|
| Prior hormonotherapy | 16 | (100) | 49 | (100) | 62 | (100) |
| Prior immunotherapy | 1 | (6.3) | 0 | | 1 | (1.6) |
| Prior targeted therapy | 13 | (81.3) | 35 | (71.4) | 45 | (72.6) |
| Other | 1 | (6.3) | 1 | (2.0) | 2 | (3.2) |

Number of prior lines in advanced settings

| | | | | | | |
|---|---|---|---|---|---|---|
| Mean (SD) | 3.2 | (1.7) | 2.5 | (1.2) | 2.6 | (1.4) |

| | | | |
|---|---|---|---|
| Median | 2.5 | 2.0 | 2.0 |
| Min; Max | 1; 8 | 1; 6 | 1; 8 |

Number of prior lines in advanced settings (by class)

| | | | | | | |
|---|---|---|---|---|---|---|
| 1 | 1 | (6.3) | 14 | (28.6) | 15 | (24.2) |
| 2 | 7 | (43.8) | 11 | (22.4) | 17 | (27.4) |
| 3 | 1 | (6.3) | 14 | (28.6) | 14 | (22.6) |
| >3 | 7 | (43.8) | 10 | (20.4) | 16 | (25.8) |

Intent of prior anti-cancer therapy

| Advanced only | 7 | (43.8) | 18 | (36.7) | 24 | (38.7) |
|---|---|---|---|---|---|---|
| Neoadjuvant and advanced | 0 | | 1 | (2.0) | 1 | (1.6) |
| Adjuvant and advanced | 7 | (43.8) | 23 | (46.9) | 28 | (45.2) |
| Neoadjuvant and adjuvant and advanced | 2 | (12.5) | 7 | (14.3) | 9 | (14.5) |

Type of prior therapy in advanced settings

| mTOR inhibitors | 7 | (43.8) | 16 | (32.7) | 21 | (33.9) |
|---|---|---|---|---|---|---|
| Anti CDK 4/6 | 12 | (75.0) | 30 | (61.2) | 39 | (62.9) |
| Anti-HER2 | 1 | (6.3) | 0 | | 1 | (1.6) |
| PI3K inhibitors | 0 | | 6 | (12.2) | 6 | (9.7) |
| Aromatase inhibitors | 15 | (93.8) | 47 | (95.9) | 59 | (95.2) |
| Antiangiogenic | 0 | | 1 | (2.0) | 1 | (1.6) |
| SERD | 9 | (56.3) | 22 | (44.9) | 29 | (46.8) |
| SERM | 5 | (31.3) | 14 | (28.6) | 18 | (29.0) |

Exposure, Safety:

Regarding exposure to the studied drug, the median duration of treatment was 23.6 weeks (range 4-90 weeks). Half of patients received ≥5 cycles of study treatment. One (1) patient had dose reduction at 400 mg dose level and six (6) patients had at least one temporary dose omission. All patients have currently discontinued the study treatment due to progressive disease.

All patients experienced at least one TEAE (all grades), regardless the relationship with the Investigational Medicinal Product (compound (1)): the most frequently reported TEAEs (in at least 2 patients) were hot flush, nausea, diarrhea and constipation, decreased appetite in 6 patients each (37.5%), fatigue, urinary tract infection, asthenia, arthralgia, dyspnoea in 4 patients each (25.0%), abdominal pains, hypoaesthesia and night sweats in 3 patients each (18.8%).

Most of these TEAEs were of grade 1 and 2. Four patients had at least one grade ≥3 event, however not related to compound (1) intake. Most frequently reported TEAEs specifically related to the Investigational Medicinal Product were as follows: hot flush (n=5; 31.3%), diarrhea, nausea (n=4 each; 25%), as well as decreased appetite, constipation, night sweats, asthenia (n=3 each; 18.8%), arthralgia and fatigue (n=2 each; 12.5%).

No DLT was observed during DLT observation period, no AE met DLT criteria definition in subsequent cycles and maximum tolerated dose (MTD) was not reached.

A total of 4 serious TEAEs have been reported in 3 patients, all considered to be not associated with compound (1) but associated to disease progression: Grade 3 back pain and Grade 5 disease progression in a patient treated at 150 mg QD, Grade 3 dyspnea in one patient treated at 200 mg QD and Grade 3 fatigue in one patient treated at 600 mg QD.

Table 2 describes all grades TEAEs (number (%) of patients with TEAE with incidence >10%) and grade ≥3 (“Gr3”), regardless of relationship to study treatment and TEAEs related to study treatment (“Related TEAEs”).

A total of 4 patients died during study period, 3 for disease progression and one with unknown reason.

All laboratory abnormalities reported as adverse events (leading dose modification/SAE) were not considered related to the compound (1) intake: 1 neutropenia (150 mg) and 1 alkaline phosphatase increase (600 mg).

Hematological laboratory abnormalities of grade 3 and 4 were limited: Grade 4 in 1 patient with lymphopenia (150 mg); Grade 3: in 2 patients with lymphopenia (200 and 600 mg), in 1 patient with leukopenia (200 mg), in 1 patient with neutropenia (150 mg) and 2 patients with anemia (150 and 600 mg).

Other grade 3 and 4 laboratory abnormalities were as follows: Grade 4 in 1 patient with AST increase (600 mg), in 1 patient with bilirubin increase (600 mg); Grade 3 in 1 patient with hyponatremia (150 mg), in 1 patient with hypoalbuminemia (600 mg), in 1 patient with AST increase (200 mg), in 1 patient with ALT increase (600 mg), in 2 patients with ALK increase (200 and 600 mg).

TABLE 2
TED14856 - Overview of adverse event profile: Number of patients with all
TEAEs with incidence >10% and related TEAEs - Safety population - Part A

| | TEAEs | | | | Related TEAEs | | |
|---|---|---|---|---|---|---|---|
| | Patients with | | Patients with | | Patients with | | Patients with |
| MedDRA PT | TEAE n(%) | | Gr3 TEAE n(%) | | TEAE n(%) | | Gr3 TEAE n(%) |
| ALL | 16 | (100) | 4 | (25.0) | 14 | (87.5) | 0 |
| Hot flush | 6 | (37.5) | 0 | | 5 | (31.3) | 0 |
| Diarrhea | 6 | (37.5) | 0 | | 4 | (25.0) | 0 |
| Constipation | 6 | (37.5) | 0 | | 3 | (18.8) | 0 |
| Nausea | 6 | (37.5) | 0 | | 4 | (25.0) | 0 |
| Decreased appetite | 6 | (37.5) | 0 | | 3 | (18.8) | 0 |
| Asthenia | 4 | (25.0) | 0 | | 3 | (18.8) | 0 |
| Fatigue | 4 | (25.0) | 1 | (6.3) | 2 | (12.5) | 0 |
| Hypoaesthesia | 3 | (18.8) | 0 | | 0 | | 0 |
| Arthralgia | 4 | (25) | 0 | | 2 | (12.5) | 0 |
| Urinary tract infection | 4 | (25) | 1 | (6.3) | 0 | | 0 |
| Dyspnoea | 4 | (25) | 1 | (6.3) | 1 | (6.3) | 0 |
| Night sweat | 3 | (18.8) | 0 | | 3 | (18.8) | 0 |
| Nasopharyngitis | 2 | (12.5) | 0 | | 0 | | 0 |
| Upper respiratory tract infection | 2 | (12.5) | 0 | | 0 | | 0 |
| Dysgeusia | 2 | (12.5) | 0 | | 0 | | 0 |
| Photophobia | 2 | (12.5) | 0 | | 1 | (6.3) | 0 |
| Cough | 2 | (12.5) | 0 | | 0 | | 0 |
| Abdominal pain upper | 2 | (12.5) | 0 | | 1 | (6.3) | 0 |
| Vomiting | 2 | (12.5) | 0 | | 1 | (6.3) | 0 |
| Dry skin | 2 | (12.5) | 0 | | 1 | (6.3) | 0 |
| Rash | 2 | (12.5) | 0 | | 0 | | 0 |
| Back pain | 2 | (12.5) | 1 | (6.3) | 1 | (6.3) | 0 |

| Myalgia | 2 | (12.5) | 0 | 1 | (6.3) | 0 |
|---|---|---|---|---|---|---|
| Musculoskeletal chest pain | 2 | (12.5) | 0 | 0 | | 0 |
| Pain | 2 | (12.5) | 0 | 1 | (6.3) | 0 |
| Hypertension | 2 | (12.5) | 0 | 1 | (6.3) | 0 |

Efficacy:

All 16 patients were evaluable as per RECISTv.1.1, and the Best Overall Response (BOR) was as follows (Table 3):
1 patient (6.3%), treated at 150 mg QD, had Partial Response (PR),
8 patients (50%) had Stable Disease (SD), and
7 patients (43.8%) had Progressive Disease.

Objective response was observed in 1 patient (ORR=6.3%). Clinical benefit (CR (Complete Response)+PR+SD≥24 weeks) was observed in 8 patients (CBR=50.0%).

In addition, tumor shrinkage greater than 10% was also observed in 9 patients.

TABLE 3
TED14856 - Best overall response - Efficacy population - Part A

| | Compound (1) | | | | | |
|---|---|---|---|---|---|---|
| | 20 mg | 150 mg | 200 mg | 400 mg | 600 mg | All |
| Number [n(%)] | (N = 3) | (N = 3) | (N = 4) | (N = 3) | (N = 3) | (N = 16) |
| Best Overall Response | | | | | | |
| Complete Response CR) | 0 | 0 | 0 | 0 | 0 | 0 |
| Partial Response (PR) | 0 | 1 (33.3) | 0 | 0 | 0 | 1 (6.3) |
| Stable Disease (SD) | 1 (33.3) | 1 (33.3) | 3 (75.0) | 2 (66.7) | 1 (33.3) | 8 (50.0) |
| Progressive Disease (PD) | 2 (66.7) | 1 (33.3) | 1 (25.0) | 1 (33.3) | 2 (66.7) | 7 (43.8) |
| Objective Response Rate | 0 | 1 (33.3) | 0 | 0 | 0 | 1 (6.3) |
| 90% CI | NC | (1.7%, 86.5%) | NC | NC | NC | (0.3%, 26.4%) |
| Clinical Benefit Rate | 0 | 2 (66.7) | 3 (75.0) | 2 (66.7) | 1 (33.3) | 8 (50.0) |
| 90% CI | NC | (13.5%, 98.3%) | (24.9%, 98.7%) | (13.5%, 98.3%) | (1.7%, 86.5%) | (27.9%, 72.1%) |

CBR was 45.5% (5/11) in patients with ESR1 mutations and 60.0% (3/5) in patients with wild-type ESR1.

Pharmacokinetics:

The pharmacokinetic (PK) profiles obtained from a total of 16 patients in fasting condition on Day 1 and Day 22 at Cycle 1 after once a day administration from 20 to 600 mg generally showed a rapid absorption (median time to achieve maximum concentration (tₘₐₓ)˜3 h) followed by a biphasic elimination profile with plasma concentrations above the Lower Limit of Quantification (LLOQ, 100 ng/mL) over the entire dosing interval for doses above 20 mg once daily.

PK parameters obtained after single and repeated administrations are presented in tables 4a and 4b below.

A high total variability of exposure parameters has been observed after single dose administration (average coefficient of variation CV >60%) and moderate variability after repeated administrations (average CV ˜45%).

Overall across the 20-600 mg range, mean apparent volume of distribution is large (˜120 L) and mean apparent systemic clearance is low (˜14-15 L/h). The mean apparent terminal half-life estimated over 48 hours after a single dose on Day 1 is of at least ˜8 hours.

A moderate accumulation is observed on Day 21/22 after once-daily administrations up to 200 mg while no accumulation was observed at higher doses.

Exposure (Cₘₐₓ, AUC₀₋₂₄ₕ) increase did not deviate significantly from dose proportionality up to 600 mg after single or multiple once daily administrations of compound (1). Average C_{trough} reached after repeated 400 mg QD was 388 ng/mL (CV: 84.2%).

Food intake (moderate-fat breakfast) did not result in a major effect on exposure, regardless of dose level; overall across doses, exposure increased by about 45% and median tₘₐₓ was delayed by approximately 1 hour.

TABLE 4a
Compound (1) plasma pharmacokinetic parameters (48 h PK profiles) following a single
administration of compound (1) (Cycle1 Day 1) under fasted condition in patients

Mean ± SD

| (Geometric Mean) | Plasma compound (1) | | | | |
|---|---|---|---|---|---|
| [CV %] | 20 mg | 150 mg | 200 mg | 400 mg | 600 mg |
| N | 3 | 3 | 4 | 3 | 3 |
| tₘₐₓ^{a} | 1.52 | 3.00 | 2.98 | 3.00 | 3.03 |
| (hr) | (1.50-2.00) | (3.00-24.77) | (1.98-4.02) | (1.50-3.83) | (2.02-4.00) |
| Cₘₐₓ | 187 ± 46.7 | 1310 ± 1380 | 1650 ± 1340 | 4740 ± 2920 | 7010 ± 4180 |
| (ng/mL) | (183) [24.9] | (884) [105.3] | (1340) [81.4] | (4120) [61.5] | (6160) [59.6] |
| AUC₀₋₂₄ | 1040 ± 560 | 9140 ± 8730 | 13600 ± 13800 | 40400 ± 17500 | 61100 ± 36300 |
| (ng · hr/mL) | (924) [53.8] | (6860) [95.6] | (9760) [101.3] | (37600) [43.2] | (53400) [59.5] |
| CL/F | 12.2 ± NC | 7.26 ± NC | 24.2 ± 17.0 | 9.86 ± 5.77 | 15.2 ± NC |
| (L/hr) | (12.2) [NC]^{b} | (7.26) [NC]^{b} | (18.8) [70.3] | (8.85) [58.6] | (14.1) [NC]^{c} |
| Vₛₛ/F | 81.9 ± NC | 61.5 ± NC | 213 ± 127 | 117 ± 40.6 | 137 ± NC |
| (L) | (81.9) [NC]^{b} | (61.5) [NC]^{b} | (173) [59.6] | (112) [34.8] | (132) [NC]^{c} |
| t_{1/2z} | 4.86 ± NC | 6.42 ± NC | 7.41 ± 1.45 | 10.6 ± 2.33 | 9.45 ± NC |
| (hr) | (4.86) [NC]^{b} | (6.42) [NC]^{b} | (7.31) [19.5] | (10.4) [22.0] | (8.97) [NC]^{c} |

^{a}Median (Min-Max)
^{b}N = 1
^{c}N = 2
NA = Not Applicable
NC: Not Calculated
SD: Standard Deviation
CV: Coefficient of Variation

TABLE 4b
Compound (1) plasma pharmacokinetic parameters following repeated once daily administrations
of compound (1) (Cycle1 Day 22) under fasted condition in patients

Mean ± SD

| (Geometric Mean) | Plasma compound (1) | | | | |
|---|---|---|---|---|---|
| [CV %] | 20 mg | 150 mg | 200 mg ^{b} | 400 mg | 600 mg |
| N (number of patients) | 3 | 2 | 3 | 3 | 3 |
| tₘₐₓ^{a} | 2.17 | 2.97 | 2.07 | 2.95 | 3.00 |
| (hr) | (1.92-4.02) | (2.93-3.00) | (2.00-3.03) | (2.02-3.78) | (3.00-4.00) |
| Cₘₐₓ | 218 ± 95.3 | 2390 ± NC | 2150 ± 873 | 4020 ± 2460 | 5570 ± 962 |
| (ng/mL) | (203) [43.7] | (2230) [NC] | (2030) [40.7] | (3370) [61.3] | (5510) [17.3] |
| AUC₀₋₂₄ | 1630 ± 1120 | 15900 ± NC | 13900 ± 4380 | 36800 ± 23500 | 42700 ± 11200 |
| (ng · hr/mL) | (1350) [68.8] | (15000) [NC] | (13400) [31.6] | (29500) [63.8] | (41700) [26.2] |
| CLₛₛ/F | 18.0 ± 13.7 | 10.6 ± NC | 15.3 ± 4.30 | 18.1 ± 17.4 | 14.7 ± 3.96 |
| (L/hr) | (14.8) [75.8] | (9.99) [NC] | (14.9) [28.0] | (13.5) [95.7] | (14.4) [26.9] |
| C_{trough} | 8.10 ± 7.16 | 126 ± NC | 76.1 ± 56.0 | 388 ± 327 | 348 ± 197 |
| (ng/mL) | (NC) [88.4] | (115) [NC] | (59.2) [73.6] | (253) [84.2] | (314) [56.8] |

^{a}Median (Min-Max)
^{b} Profile of one Subject was excluded (N = 3)
NC: Not Calculated
SD: Standard Deviation
CV: Coefficient of Variation

### Pharmacodynamics (PD) and PK/PD:

Pharmacodynamics was assessed using ¹⁸FES-PET scans; 14 patients had one examination at baseline and one between 11 and 15 days after the first administration of compound (1), with the PET scan being close to the time of PK trough prior to the next dose. Of the remaining 2 patients, one had the on-treatment ¹⁸FES-PET on Day 10, and the other had it on Day 28: these were deviations, and the decision was made to include in the PD analysis (and dose escalation decision) the patient who had the ¹⁸FES-PET on D10, which was an evaluation after at least 8 days of continuous treatment.

Because at least one patient out of 3 treated at the 20 mg dose level had ¹⁸FES-PET scan results showing less than 30% inhibition of the target, the 100 mg dose level was skipped (as per protocol). High levels of inhibition of the signal were observed starting from the 150 mg dose level and up: 100% inhibition at this dose level and 65-100% in all other patients treated at further dose levels (see Table 4c).

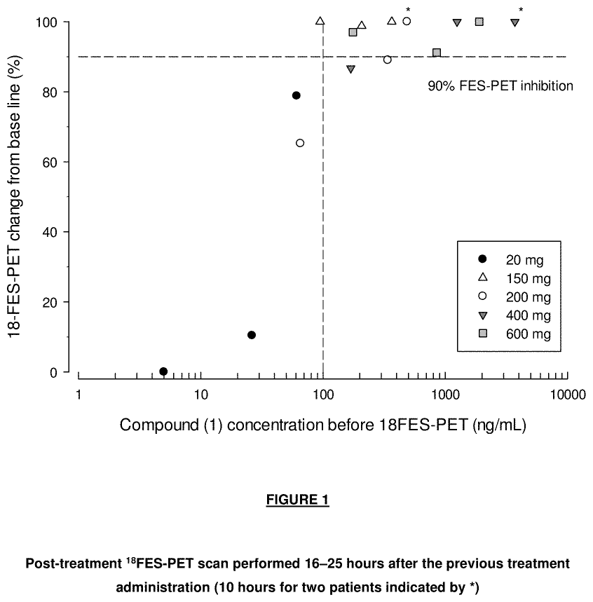

A strong PK/PD relationship was established between plasma concentrations of compound (1) measured just before ¹⁸FES administration and concomitant inhibition of ¹⁸FES-PET signal. The ¹⁸FES-PET inhibition generally exceeded 87%, close to 90% as specified in the protocol, was generally observed when plasma concentrations were above 100 ng/mL (see FIG. 1). This threshold is thus expected to correlate with high occupancy of ERs by compound (1).

TABLE 4c
¹⁸FES-PET results by dose levels

| Dose levels | Nb of patients | ¹⁸FES-PET % of occupancy (reduction) |
|---|---|---|
| 20 mg | 3 | 0%-10.4%-78.8% |
| 150 mg | 3 | 98.8%-100%-100% |
| 200 mg | 4 | 65.2% ^{(a)}-89%-96.4% ^{(b)}-100% |
| 400 mg | 3 | 86.7%-100%-100% |
| 600 mg | 3 | 91.2%-97%-100% |

^{(a)} Low value explained by the low exposure of this patient (see FIG. 1, about 65 ng/mL)
^{(b)} ¹⁸FES-PET done outside protocol permitted window, not taken into account in decision process.

Conclusion for Study Part A:

Overall compound (1) was well tolerated, with a favorable safety profile at all tested dose levels. Adverse events (AE) observed were generally of grade 1 or 2. No dose-limiting toxicity (DLT) was observed during cycle 1 neither AE meeting DLT criteria in subsequent cycles.

Compound (1) showed high ER occupancy, with results of ¹⁸FES-PET scans indicating almost 90% or more occupancy of ERs starting from the 150 mg dose level and up to the 600 mg dose level.

Preliminary antitumor activity was encouraging, in both ESR1 mutated and wild-type patients. However, these observations of encouraging signs of activity remain to be confirmed in the dose expansion phase, as Part A of the study is mainly designed for selecting the recommended dose and for safety assessments.

Pharmacokinetics of compound (1) indicated limited accumulation and dose proportional increase of exposure up to 600 mg after repeated oral administration. At this dose mean C_{trough} concentrations after repeated administrations were well above the minimum concentration, allowing 90% occupancy of ERs.

The impact of food was minimal and compound (1) can be administered with or without food.

As a conclusion, with no DLTs and no MTD observed in the study, the 150 to 600 mg doses are assessed as suitable for treating cancer patients as defined above. The dose of 400 mg QD was selected for expansion cohorts.

3: Results for Part B of the Study

Patients Characteristics:

Dose expansion at 400 mg QD monotherapy (Part B) is ongoing with a total of 49 patients being treated. At the cut-off date of 31 Mar. 2020, five (5) patients (10.2%) remained on study treatment. The mean age was 63.1 (±10.6) years. The ECOG status was 1 in 40.8% of the patients and ECOG 0 in the remaining patients. The median time from first diagnosis to first study treatment administration was 6.04 years (range 0.8 to 24.3).

All patients were ER− positive and HER2− negative. 72.3% of the patients were progesterone receptor positive.

All patients were metastatic. They had a median of 3 organs involved at study entry (range 1 to 6). The main organs involved were bone (71.4%) and liver (57.1%) followed by lymph nodes (38.8%), lung (30.6%) and breast (24.5%).

Patients were heavily pretreated, receiving several lines of anti-cancer treatments, including chemotherapy, hormonal therapy and targeted therapy, with a median of 2 prior anti-cancer therapies (range 1-6) in the advanced setting; 95.9% received prior aromatase inhibitor, 61.2% were treated with a CDK 4/6 inhibitor, 44.9% received prior SERD-based therapy, 32.7% mTOR inhibitor and 28.6% SERM.

The demographic and the baseline disease characteristics of the patients treated in Part B of TED14856 study are provided in Tables 1a and 1b above.

Safety:

Regarding the exposure to the studied drug, the median duration of treatment was 10.1 weeks (range 1-69 weeks). A total of 40.8% of patients received ≥5 cycles of study treatment. Four (4) patients had dose reduction and fourteen (14) patients had at least one temporary dose omission. A total of 44 patients (89.8%) have discontinued the study treatment including 41 (83.7%) due to progressive disease, 1 (2%) for TEAE and 2 (4.1%) for other reasons.

All patients experienced at least one TEAE (all grades), regardless the relationship with the Investigational Medicinal Product (compound (1)): the most frequently reported TEAEs (>12%) were: vomiting and constipation (26.5%), fatigue (24.5%), abdominal pain (22.4%), nausea (20.4%), asthenia and arthralgia (18.4%), dyspnea and diarrhea (16.3%), hot flush and decreased appetite (14.3%) (Table 5).

Most of these TEAEs were of grade 1 and 2. A total of 15/49 (30.6%) patients had at least one grade ≥3 event, however none of them were related to compound (1) intake.

Most frequently reported TEAEs (in at least 2 patients) specifically related to the Investigational Medicinal Product were as follows: hot flush (10.2%), vomiting and arthralgia (8.2%), constipation and gastroesophageal reflux disease (6.1%), fatigue, depression, nausea, abdominal pain and decreased appetite (4.1%).

A total of thirteen patients (26.5%) had at least one Serious Adverse Event. All of them were not considered related to the compound (1) intake. The large majority (11 patients, 22.4%) presented with 14 SAEs of grade ≥3: 2 occurrences of pulmonary embolism; one occurrence of the following SAES, pneumonia, breast cellulitis, cellulitis, tumor pain, hypercalcemia, vomiting, nausea, hyperbilirubinemia, jaundice cholestatic, spinal pain, disease progression and death.

Table 5 describes all grades (number (%) of patients with TEAE with incidence >5%) and grade ≥3 TEAEs, regardless of relationship to study treatment and TEAEs related to study treatment (“Related TEAEs”).

A total of 4 patients (8.2%) died: two patients during study treatment period, 1 for non-related adverse event (pneumonia) and 1 for unknown reason, the two other patients died during the study follow-up period due to disease progression.

TABLE 5
TED14856 - Overview of adverse event profile: Number of patients with all
TEAEs with incidence >5% and related TEAEs - Safety population - Part B

| | TEAEs | | | | Related TEAEs | | |
|---|---|---|---|---|---|---|---|
| | Patients with | | Patients with | | Patients with | | Patients with |
| MedDRA PT | TEAE n(%) | | Gr3 TEAE n(%) | | TEAE n(%) | | Gr3 TEAE n(%) |
| ALL | 49 | (100) | 15 | (30.6) | 26 | (53.1) | 0 |
| Constipation | 13 | (26.5) | 0 | | 3 | (6.1) | 0 |
| Vomiting | 13 | (26.5) | 1 | (2.0) | 4 | (8.2) | 0 |
| Fatigue | 12 | (24.5) | 2 | (4.1) | 2 | (4.1) | 0 |
| Abdominal pain | 11 | (22.4) | 0 | | 2 | (4.1) | 0 |
| Nausea | 10 | (20.4) | 1 | (2.0) | 2 | (4.1) | 0 |

| Arthralgia | 9 | (18.4) | 0 | 4 | (8.2) | 0 |
|---|---|---|---|---|---|---|
| Asthenia | 9 | (18.4) | 0 | 1 | (2.0) | 0 |
| Diarrhoea | 8 | (16.3) | 0 | 1 | (2.0) | 0 |
| Dyspnoea | 8 | (16.3) | 0 | 1 | (2.0) | 0 |
| Hot flush | 7 | (14.3) | 0 | 5 | (10.2) | 0 |
| Decreased appetite | 7 | (14.3) | 0 | 2 | (4.1) | 0 |
| Abdominal pain upper | 5 | (10.2) | 0 | 1 | (2.0) | 0 |
| Back pain | 5 | (10.2) | 0 | 0 | | 0 |
| Urinary tract infection | 5 | (10.2) | 0 | 1 | (2.0) | 0 |
| Nasopharyngitis | 4 | (8.2) | 0 | 0 | | 0 |
| Gastroesophageal reflux disease | 4 | (8.2) | 0 | 3 | (6.1) | 0 |
| Cough | 4 | (8.2) | 0 | 1 | (2.0) | 0 |
| Pyrexia | 4 | (8.2) | 0 | 0 | | 0 |
| Hypertension | 4 | (8.2) | 0 | 1 | (2.0) | 0 |
| Headache | 3 | (6.1) | 0 | 0 | | 0 |
| Depression | 3 | (6.1) | 0 | 2 | (4.1) | 0 |
| Rhinitis allergic | 3 | (6.1) | 0 | 0 | | 0 |
| Musculoskeletal pain | 3 | (6.1) | 0 | 1 | (2.0) | 0 |
| Dry skin | 3 | (6.1) | 0 | 1 | (2.0) | 0 |

| | | | | | | |
|---|---|---|---|---|---|---|
| ALT increased | 3 | (6.1) | 3 | (6.1) | 0 | 0 |
| AST increased | 3 | (6.1) | 3 | (6.1) | 0 | 0 |

All laboratory abnormalities reported as adverse events (leading to dose modification/SAE) were not considered related to the compound (1) intake: 3 increases of alanine amino transferase and of aspartate amino transferase, 1 hypercalcemia, 2 hyperbilirubinemia and 1 alkaline phosphatase increase.

Hematological laboratory abnormalities of grade 3 and 4 were limited: Grade 3 in 5 patients with lymphopenia, in 1 patient with neutropenia and 1 with anemia; none of them were reported as adverse event.

Other grade 3 and 4 laboratory abnormalities were as follows: Grade 4 in 1 patient with hyponatremia, in 1 patient with hypokalemia, in 1 patient with hypocalcemia; Grade 3 in 2 patients with hyponatremia, in 1 patient with hypercalcemia, in 2 patients with hypophosphatemia, in 6 patients with AST increase, in 4 patients with ALT increase, in 2 patients with ALK increase and in 3 patients with bilirubin increase.

Efficacy:

A total of 46/49 (93.9%) patients were assessed as per RECISTv.1.1, and the Best Overall Response (BOR) assessed by an Independent Central Review (ICR) was as follows (Table 6):
3 patients (6.5%) had Partial Response (PR),
21 patients (45.7%) had Stable Disease (SD),
20 patients (43.5%) had Progressive Disease, and
2 were not evaluable.

Objective response was observed in 3 patients (ORR=6.5%). Clinical benefit (CR (Complete Response)+PR+SD≥24 weeks) was observed in 12 patients (CBR=26.1%). In addition, tumor shrinkage greater than 10% was also observed in 13 (28.3%) patients.

TABLE 6
TED14856 - Best overall response assessed by Independent
Central Review - Efficacy population - Part B

| | Compound (1) 400 mg | |
|---|---|---|
| | (N = 46) | |
| Best Overall Response [n(%)] | | |
| Number | 46 | |
| Complete Response (CR) | 0 | |
| Partial Response (PR) | 3 | (6.5) |
| Stable Disease (SD) | 21 | (45.7) |
| Progressive Disease (PD) | 20 | (43.5) |
| Not Evaluable (NE) | 2 | (4.3) |
| Objective Response Rate (confirmed CR and PR) [n(%)] | 3 | (6.5) |
| 90% CI | (1.8%, 16.0%) | |
| Clinical Benefit Rate [n(%)] | 12 | (26.1) |

| | |
|---|---|
| 90% CI | (15.8%, 38.8%) |

The Clinical Benefit Rate (CBR, corresponding to CR+PR+SD≥24 weeks) assessed by ICR was 15.8% (3/19) in ESR1 mutated patients and 34.6% (9/26) in patients with wild-type ESR1.

4: Pooled Results for Part A (for Patients Receiving a Dose of Study Treatment Equal to or Greater than 150 mg) and Part B of the Study (“Pooled Population”)

Post-hoc analyses have been performed by pooling patients from Part A patients receiving a dose of study treatment equal to or greater than 150 mg (150-600 mg range) and Part B patients at 400 mg. Efficacy analyses were performed in order to assess the activity of compound (1) on the 59 response-evaluable patients treated with active doses of the compound at the cut-off date of 31 Mar. 2020, meaning 13 patients of Part A (after exclusion of the 20 mg dose level QD) and 46 patients treated at 400 mg of Part B.

It is important to note the heterogeneity of this population regarding the number of prior lines of therapy and particularly the targeted therapies such as mTOR and CDK4/6 inhibitors that have been recently approved and their impact on the assessment of activity of compound (1). While data on response after progression to hormonal therapy are available, very few data exist after patients progressed to these new targeted agents or after several lines of therapies. Therefore, these analyses aim to assess the activity of compound (1) in subpopulations depending on prior therapies and on the number of prior lines the patients have taken.

The demographic and the baseline disease characteristics of the patients treated in the pooled population (N=62) of TED14856 study are provided in Tables 1a and 1b above.

A total of 59 patients were assessed as per RECISTv.1.1, the Best Overall Response (BOR) by investigator was as follows (Table 7):
4 patients (6.8%) had Partial Response (PR),
25 patients (42.4%) had Stable Disease (SD),
30 patients (50.8%) had Progressive Disease.

Objective response was observed in 4 patients (ORR=6.8%). Clinical Benefit (CR (Complete Response)+PR+SD≥24 weeks) was observed in 21 patients (CBR=35.6%).

TABLE 7
TED14856 - Efficacy analyses of compound (1) - Pooled Population

| | Compound (1) >=150 mg | | Compound (1) 400 mg | | | |
|---|---|---|---|---|---|---|
| | PART A | | PART B | | All | |
| | (N = 13) | | (N = 46) | | (N = 59) | |
| Best Overall Response [n(%)] | | | | | | |
| Complete Response (CR) | 0 | | 0 | | 0 | |
| Partial Response (PR) | 1 | (0.7) | 3 | (6.5) | 4 | (6.8) |
| Stable Disease (SD) | 7 | (53.8) | 18 | (39.1) | 25 | (42.4) |
| Progressive Disease (PD) | 5 | (38.5) | 25 | (54.3) | 30 | (50.8) |
| Objective Response Rate | 1 | (77) | 3 | (6.5) | 4 | (6.8) |
| (confirmed CR and PR) [n(%)] | | | | | | |
| 90% CI | (0.4%, 31.6%) | | (1.8%, 16.0%) | | (2.3%, 14.8%) | |
| Clinical Benefit Rate [n(%)] | 8 | (61.5) | 13 | (28.3) | 21 | (35.6) |

| | | | |
|---|---|---|---|
| 90% CI | (35.5%, 83.4%) | (17.6%, 41.1%) | (25.2%, 47.1%) |

The Clinical Benefit Rate (CBR, corresponding to CR+PR+SD≥24 weeks) was 32.1% (9/28) in ESR1 mutated patients and 40.0% (12/30) in patients with wild-type ESR1.

To evaluate the activity of compound (1), different sub-population analyses have been performed, as it is described in detail below.

A comparison of the results observed in the present clinical study (TED14856) with those of fulvestrant in the literature was undertaken in order to indirectly compare the results from compound (1) with those published from fulvestrant. A total of 4 comparative studies have been selected with fulvestrant at either 250 mg or 500 mg. The efficacy results (ORR and CBR) were consistent between studies in the fulvestrant single agent arms with an ORR of about 7 to about 12% and a CBR of 31 to about 37% (see Table 8).

In the pooled population analysis (i.e., in more heavily pretreated patients, with pretreatments including targeted therapy and/or fulvestrant), indirect comparison with the literature shows similar efficacy results to those of fulvestrant studies, with an ORR of 6.8% and a CBR of 35.6% (Tables 7 and 8).

In general, the assessment of activity or efficacy of a compound is performed, as much as possible, in a homogenous population with patients who have been exposed to a “reasonable” number of lines of therapy or with a similar exposure to prior therapies. Therefore, the two following subpopulations were analyzed:
First, a subset of 32 patients selected from the pooled population of the TED14856 study, who have previously received no more than 3 treatment lines in metastatic setting and i) could have received a prior chemotherapy or a CDK4/6 inhibitor treatment but not both, and ii) have not received prior treatment with mTOR, was selected and investigated. ORR and PRs were of 12.5% (4/32 patients each), SD was of 50% (16/32 patients) and PD was of 37.5% (12/32 patients). Even though this sub-population of 32 patients was still more heavily pretreated compared to the patients of the fulvestrant arms from the 4 selected studies, indirect comparison of CBR shows a trend to higher rate with compound (1) compared to fulvestrant: 46.9% (15/32) versus 31% to about 37%, respectively (Tables 8 and 9).
Second, a sub-population of 14 patients, with prior treatments very similar to the ones according to the fulvestrant published studies, i.e. who received neither prior targeted therapies nor SERD, was identified in the TED14856 study and analyzed. In this sub-population ORR and PRs were of 21.4% (3/14 patients), SD was of 57.1% (8/14 patients) and PD was of 21.4% (3/14 patients). Overall in this population, the clinical benefit was seen in 9/14 patients, corresponding to a CBR of 64.3% (Tables 8 and 10). Indirect comparison to the fulvestrant arm from the 4 selected comparative studies shows a trend to a higher ORR and CBR with compound (1).

TABLE 8
TED14856 - Comparative studies with fulvestrant single agent

| | | | | | | Prior | Prior | Prior |
|---|---|---|---|---|---|---|---|---|
| Trial | Period | n | ORR | CBR | Setting | CDK 4/6 | mTOR | fulvestrant |
| SANDPIPER ¹ | 2015-2017 | 134 | 11.9% | 37.3% | 2L+ | Not allowed | Not allowed | Not allowed |
| PALOMA-3 ² | 2013-2014 | 138 | 11% | 36% | 2L+ | Not allowed | Not allowed | Not allowed |
| SoFEA ³ | 2004-2010 | 178 | 8% | 31% | 2L+ | Not allowed (not | Not allowed (not | Not allowed |
| | | | | | | approved at trial dates) | approved at trial dates) | |
| EFECT ⁴ | 2003-2005 | 270 | 7.4% | 32.2% | 2L+ | Not allowed (not | Not allowed (not | Not allowed |
| | | | | | | approved at trial dates) | approved at trial dates) | |
| TED14856 | 2017-2019 | 59 | 6.8% | 35.6% | (2-9L) | Allowed | Allowed | Allowed |
| | 2017-2019 | 32 | 12.5% | 46.9% | 3L | Allowed | Not allowed | Allowed |
| | 2017-2019 | 14 | 21.4% | 64.3% | 2L+ | None | None | None |

¹ Baselga J. et al., Phase III study of taselisib (GDC-0032) + fulvestrant (FULV) v FULV in patients (pts) with estrogen receptor (ER)-positive, PIK3CA-mutant (MUT), locally advanced or metastatic breast cancer (MBC): Primary analysis from SANDPIPER. Journal of Clinical Oncology, 2018; 36: 18 suppl.
² Cristofanilli M. et al., Fulvestrant plus palbociclib versus fulvestrant plus placebo for treatment of hormone- receptor-positive, HER2-negative metastatic breast cancer that progressed on previous endocrine therapy (PALOMA 3): final analysis of the multicentre, double-blind, phase 3 randomised controlled trial. Lancet Oncology, April 2016; 17(4): 425-39
³ Johnston S. R. et al., Fulvestrant plus anastrozole or placebo versus exemestane alone after progression on non-steroidal aromatase inhibitors in postmenopausal patients with hormone-receptor-positive locally advanced or metastatic breast cancer (SoFEA): a composite, multicentre, phase 3 randomised trial. Lancet Oncology, September 2013; 14(10): 989-98
⁴ Chia S. et al., Double-blind, randomized placebo controlled trial of fulvestrant compared with exemestane after prior nonsteroidal aromatase inhibitor therapy in postmenopausal women with hormone receptor positive, advanced breast cancer: Results from EFECT. Journal of Clinical Oncology, 2008; 26(10): 1664-70

TABLE 9
Exploratory efficacy analyses in patients in advanced settings - Pooled population patients
with ≤3 prior lines in metastatic setting, without both prior chemotherapy and CDK4/6
inhibitor (none or one of them is allowed) and without prior mTOR inhibitor.

| | Compound (1) ≥150 | Compound (1) 400 mg | | | |
|---|---|---|---|---|---|
| | mg PART A | PART B | | All | |
| Exploratory efficacy analyses | (N = 6) | (N = 26) | | (N = 32) | |
| Best Overall Response [n(%)] | | | | | |
| Complete Response (CR) | 0 | 0 | | 0 | |
| Partial Response (PR) | 1 (16.7) | 3 | (11.5) | 4 | (12.5) |
| Stable Disease (SD) | 3 (50.0) | 13 | (50.0) | 16 | (50.0) |
| Progressive Disease (PD) | 2 (33.3) | 10 | (38.5) | 12 | (37.5) |
| Objective Response Rate (confirmed CR and PR) [n(%)] | 1 (16.7) | 3 | (11.5) | 4 | (12.5) |
| 90% Cl | (0.9%, 58.2%) | (3.2%, 27.2%) | | (4.4%, 26.4%) | |
| Clinical Benefit Rate [n(%)] | 4 (66.7) | 11 | (42.3) | 15 | (46.9) |

| | | | |
|---|---|---|---|
| 90% Cl | (27.1%, 93.7%) | (25.8%, 60.2%) | (31.5%, 62.7%) |

TABLE 10
TED14856 - Exploratory efficacy analyses in patients in advanced settings without
prior mTOR, prior CDK4/6 and prior fulvestrant - Pooled Population (n = 14)

| | Compound (1) ≥150 mg | Compound (1) 400 mg | | | |
|---|---|---|---|---|---|
| | PART A | PART B | | All | |
| Exploratory efficacy analyses | (N = 3) | (N = 11) | | (N = 14) | |
| Best Overall Response [n(%)] | | | | | |
| Complete Response (CR) | 0 | 0 | | 0 | |
| Partial Response (PR) | 0 | 3 | (27.3) | 3 | (21.4) |
| Stable Disease (SD) | 2 (66.7) | 6 | (54.5) | 8 | (57.1) |
| Progressive Disease (PD) | 1 (33.3) | 2 | (18.2) | 3 | (21.4) |
| Overall Response Rate (confirmed CR and PR) [n(%)] | 0 | 3 | (27.3) | 3 | (21.4) |
| 90% CI | NC | (7.9%, 56.4%) | | (6.1%, 46.6%) | |
| Clinical Benefit Rate [n(%)] | 2 (66.7) | 7 | (63.6) | 9 | (64.3) |

| | | | |
|---|---|---|---|
| 90% CI | (13.5%, 98.3%) | (35.0%, 86.5%) | (39.0%, 84.7%) |

### CONCLUSION

There is currently no pivotal or relevant comparative study demonstrating efficacy of an

ET (Endocrine Therapy), whether as single agent or in combination with a targeted therapy, after progression to CDK4/6 inhibitor combined with an ET. A significant unmet medical need remains in this population that requires novel therapy like new SERD compounds, such as compound (1) described herein, to be added to the oral treatment administration options.

The study described herein showed that compound (1) was well tolerated in the metastatic breast cancer patients, with a favorable safety profile at all tested dose levels from 20 to 600 mg. Adverse events (AE) observed were generally of grade 1 or 2.

Compound (1) showed high ER occupancy, with results of ¹⁸FES-PET scans indicating almost 90% or more occupancy of ERs starting from the 150 mg dose level and up to the 600 mg dose level.

Pharmacokinetics of compound (1) indicated limited accumulation and dose proportional increase of exposure up to 600 mg after repeated oral administration. At this dose mean C_{trough} concentrations after repeated administrations were well above the minimum concentration, allowing 90% occupancy of ERs. The impact of food was minimal and compound (1) can be administered with or without food.

With no DLTs and no MTD observed in the study, the 150 to 600 mg doses are assessed as suitable for treating cancer patients as defined above. The dose of 400 mg QD single agent was selected for expansion cohorts.

The results of the activity observed with compound (1) at 150-600 mg QD, in a pooled population of heavily pretreated patients, show a durable control of the disease, with a high rate of long stabilization (CBR of 35.6%). In these heavily pretreated patients, an indirect comparison of ORR and CBR results to historical fulvestrant results shows a similar trend, although patients from fulvestrant studies were less heavily pre-treated (they did not receive prior targeted therapy nor fulvestrant).

Analyses of sub-populations according to the number of prior lines of therapies as well as prior targeted therapies shows a trend to a higher objective response rate, partial responses and clinical benefit rate compared to the historical fulvestrant performance, based on indirect literature comparisons.

In an indirect comparison of compound (1) to fulvestrant arms from the selected comparative studies and in a similar population of patients naïve from targeted therapies and fulvestrant, a trend to a higher ORR and CBR was observed.

In addition, the safety and tolerability profiles of compound (1) are favorable in the study presented herein, with TEAEs of low grades and no related serious adverse events.

1. A method for treating metastatic or advanced breast cancer comprising administering to a patient in need thereof a therapeutically effective amount of 150-600 mg per day of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof.

2. The method according to claim 1, wherein said dose is 400 to 600 mg per day.

3. The method according to claim 1, wherein said dose is 400 mg per day.

4. The method according to claim 1, wherein said use is for a human patient.

5. The method according to claim 1, wherein the breast cancer is an estrogen receptor positive cancer.

6. The method according to claim 1, wherein the breast cancer is a human epidermal growth factor receptor 2 negative cancer.

7. The method according to claim 1, wherein the breast cancer is an estrogen receptor positive, human epidermal growth factor receptor 2 negative cancer.

8. The method according to claim 1, wherein the cancer is metastatic.

9. The method according to claim 1, wherein the patient has been pretreated with at least one endocrine therapy treatment for advanced cancer.

10. The method according to claim 9, wherein the patient demonstrates disease progression or recurrence after said endocrine therapy.

11. The method according to claim 9, wherein the patient has been pretreated with at least one chemotherapy and/or targeted therapy such as a tyrosine kinase inhibitor.

12. The method according to claim 9, wherein the patient has been pretreated with one to three chemotherapies and/or one or more targeted therapies, with a range of 1 to 8 prior anti-cancer treatments.

13. The method according to claim 1, wherein the patient has received no more than 3 prior lines of anti-cancer therapies, has received no prior mTOR inhibitor treatment, and has optionally received a prior chemotherapy or a CDK4/6 inhibitor treatment but not both.

14. The method according to claim 1, wherein the patient has received neither prior targeted therapies, such as mTOR or CDK4/6 inhibitors, nor prior SERD therapy, such as fulvestrant.

15. The method according to claim 1, wherein the patient is a woman.

16. The method according to claim 15, wherein the patient is a postmenopausal woman.

17. The method according to claim 15, wherein the patient is a premenopausal woman on menopause-inducing medication.

18. (canceled)

19. A pharmaceutical composition comprising 150 to 600 mg of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, and at least one pharmaceutically acceptable excipient.

20. A method for treating metastatic or advanced breast cancer comprising administering to a patient in need thereof a pharmaceutical composition according to claim 19.

21. An article of manufacture, a packaging, or an administration unit, comprising:
a packaging material;
the pharmaceutical composition according to claim 19; and
a label or package insert contained within said packaging material, indicating that said pharmaceutical composition is administered to a patient for the treatment of metastatic or advanced breast cancer, at a dose of 150 to 600 mg per day of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof.

22. The method according to claim 10, wherein the patient has been pretreated with at least one chemotherapy and/or targeted therapy such as a tyrosine kinase inhibitor.

23. The method according to claim 10, wherein the patient has been pretreated with one to three chemotherapies and/or one or more targeted therapies, with a range of 1 to 8 prior anti-cancer treatments.
